(19) United States
(12) Patent Application Publication
(10) Pub. No.: US 2023/0332212 A1
(43) Pub. Date: Oct. 19, 2023

(54) COMPOSITIONS AND METHODS FOR BINDING AN ANALYTE TO A CAPTURE PROBE

(71) Applicant: 10x Genomics, Inc., Pleasanton, CA (US)

(72) Inventors: Jennifer Chew, Hayward, CA (US); Marlon Stoeckius, Stockholm (SE)

(21) Appl. No.: 18/311,644

(22) Filed: May 3, 2023

Related U.S. Application Data
(63) Continuation of application No. PCT/US2021/058290, filed on Nov. 5, 2021.
(60) Provisional application No. 63/110,749, filed on Nov. 6, 2020.

(51) Int. Cl. C12Q 1/6837; C12Q 1/48; G01N 1/30

(52) U.S. Cl. CPC C12Q 1/6837; C12Q 1/48; G01N 1/30

(57) ABSTRACT

Provided herein are methods and kits for binding of an analyte capture sequence to a capture domain of a capture probe.

### CROSS-REFERENCE TO RELATED APPLICATIONS

Pursuant to 35 U.S.C. § 119(e), this application is a continuation of International Application PCT/US2021/058290, with an international filing date of Nov. 5, 2011, which claims priority to U.S. Provisional Application Ser. No. 63/110,749, filed on Nov. 6, 2020. The disclosure of the prior application is considered part of the disclosure of this application and is incorporated herein by reference in its entirety.

### SEQUENCE LISTING

This application contains a Sequence Listing that has been submitted electronically as an XML file named “47706-0272001_SL_ST26.XML.” The XML file, created on Apr. 4, 2023, is 36,675 bytes in size. The material in the XML file is hereby incorporated by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Increasing resolution of spatial heterogeneity can be achieved by increasing capture efficiency or by reducing background signal. This is usually achieved by relying on the affinity of the capture reagents and/or optimized reaction conditions, neither of which address methods when using analyte capture agents to target an analyte. Therefore, there remains a need to develop strategies to enhance the binding of analyte capture agents to target analytes.

### SUMMARY

The present disclosure features methods and kits for spatially determining the location of analytes within a biological sample. Determining the spatial location of analytes (e.g., protein) within a biological sample leads to better understanding of spatial heterogeneity in various contexts, such as disease models. The methods and kits disclosed herein provide for enhancing the specificity of binding an analyte capture sequence to a capture domain. In some examples, an analyte capture agent includes an analyte binding moiety, an analyte capture sequence, and an analyte binding moiety barcode. In some examples, the analyte capture sequence is a nucleotide sequence. In some embodiments, the analyte capture sequence binds to a capture domain of a capture probe, where the capture probe includes a spatial barcode.

More specifically, the methods provided herein utilize blocking probes to block non-specific hybridization of an analyte capture sequence to the capture domain of a capture probe on an array thus enhancing the specificity of binding of an analyte capture sequence and a capture domain. In some examples, the blocking probes can differ in length and/or complexity. In some examples, the blocking probes specifically bind the analyte capture sequence. In some examples, the blocking probes specifically bind the capture domain of the capture probe on the substrate. In some examples, the blocking probes specifically bind both the analyte capture sequence and the capture domain of the capture probe on the substrate. In some examples, more than one blocking probe specifically binds the analyte capture sequence. In some examples, the blocking probe include one or more inosine nucleotides. In some examples, the blocking probes include one or more uracil nucleotides. In some examples, the blocking probes include one or more abasic sites. In some examples, the blocking probes are released by one or more of heat, cleavage, or washes in a salt buffer.

Also provided herein are methods for binding an analyte capture sequence to a capture domain including: (a) contacting a biological sample with an array, where the array includes a plurality of capture probes including (i) a spatial barcode and (ii) a capture domain; (b) providing a plurality of analyte capture agents, where an analyte capture agent includes an analyte binding moiety that binds to an analyte in the biological sample, an analyte binding moiety barcode, and an analyte capture sequence, where the capture domain, the analyte capture sequence, or both, are reversibly blocked with one or more blocking probes; and (c) releasing the one or more blocking probes from the capture domain, the analyte capture sequence, or both, and allowing the analyte capture sequence to specifically bind to the capture domain, thereby binding the analyte capture sequence to the capture domain.

In some embodiments, the blocking enhances the specificity of binding an analyte capture sequence to a capture domain compared to analyte binding specificity without blocking the capture domain, the analyte domain, or both.

In some embodiments, the method includes fixing the biological sample, and optionally where the fixing includes methanol, and staining the biologogical sample, and optionally, where the staining includes immunofluorescence.

In some embodiments, prior to the contacting in step (b), providing the plurality of analyte capture agents with the one or more blocking probes. In some embodiments, the capture domain is reversibly blocked with a blocking probe of the one or more blocking probes. In some embodiments, the analyte capture sequence is reversibly blocked with a blocking probe of the one or more blocking probes. In some embodiments, the capture domain is reversibly blocked with a first blocking probe of the one or more blocking probes and the analyte capture sequence is reversibly blocked with a second blocking probe of the one or more blocking probes.

In some embodiments, the releasing of the one or more blocking probes includes the use of an enzyme. In some embodiments, the enzyme is an endonuclease. In some embodiments, the one or more blocking probes includes one or more inosine nucleotides and the endonuclease is endonuclease V. In some embodiments, a blocking probe of the one or more blocking probes includes one or more abasic sites and the endonuclease is endonuclease IV.

In some embodiments, a blocking probe of the one or more blocking probes includes a uracil and the enzyme is a uracil-specific excision reagent (USER). In some embodiments, the blocking probe includes a poly(U) sequence, one or more RNA bases, one or more LNA bases, or combinations thereof.

In some embodiments, a blocking probe of the one or more blocking probes, when hybridized to the analyte capture sequence or the capture domain, includes one or more mismatched nucleotides, and the releasing includes increasing the temperature of the biological sample.

In some embodiments, one or more mismatched nucleotides in the blocking probe hybridized to the analyte capture sequence or the capture domain are positioned after the fourth nucleotide from a 5′ end of the blocking probe and before the last four nucleotides at the 3′ end of the blocking probe. In some embodiments, one or more mismatched nucleotides in the blocking probe hybridized to the analyte capture sequence or the capture domain are positioned after the sixth nucleotide from the 5′ end of the blocking probe and before the last six nucleotides at the 3′ end of the blocking probe.

In some embodiments, the blocking probe has a length of about 8 to about 24 nucleotides. In some embodiments, the releasing of the one or more blocking probes includes washing the biological sample. In some embodiments, the method includes permeabilizing the biological sample.

In some embodiments, the capture domain includes a nucleotide sequence of about 10 to 25 nucleotides in length. In some embodiments, the capture domain includes a unique nucleotide sequence.

In some embodiments, the analyte is a protein.

In some embodiments, the analyte binding moiety is an antibody or an antigen-binding fragment thereof.

In some embodiments, the analyte capture agent includes a linker, where the linker is disposed between the analyte binding moiety and the analyte binding moiety barcode. In some embodiments, the linker is a cleavable linker, and optionally, where the cleavable linker is a photo-cleavable linker or an enzyme cleavable linker.

In some embodiments, the method includes determining a sequence of (i) all or a part of the sequence of the analyte binding moiety barcode or a complement thereof, and (ii) all or a part of the sequence of the spatial barcode or a complement thereof, and using the determined sequence of (i) and (ii) to identify a location of the analyte in the biological sample. In some embodiments, the determining includes sequencing (i) all or a part of the sequence of the analyte binding moiety barcode or a complement thereof, and (ii) all or a part of the sequence of the spatial barcode or a complement thereof. In some embodiments, the sequencing includes high throughput sequencing.

In some embodiments, the biological sample is a tissue sample, a fixed tissue sample, a formalin-fixed paraffin-embedded tissue sample, or a fresh-frozen tissue sample.

Also provided herein are kits including: (a) an array, where the array includes a plurality of capture probes, where a capture probe of the plurality of capture probes includes a spatial barcode and a capture domain; and (b) a plurality of analyte capture agents, where an analyte capture agent includes an analyte binding moiety that binds specifically to an analyte in a biological sample, an analyte binding moiety barcode, and an analyte capture sequence, where the capture domain, the analyte capture sequence, or both are reversibly blocked with one or more blocking probes.

In some embodiments, the capture domain is reversibly blocked with a blocking probe of the one or more blocking probes. In some embodiments, the analyte capture sequence is reversibly blocked with a blocking probe of the one or more blocking probes. In some embodiments, the capture domain is reversibly blocked with a first blocking probe of the one or more blocking probes and the analyte capture sequence is reversibly blocked with a second blocking probe of the one or more blocking probes.

In some embodiments, the kit includes an enzyme. In some embodiments, the enzyme is an endonuclease. In some embodiments, a blocking probe of the one or more blocking probes includes one or more inosine nucleotides and the endonuclease is endonuclease V. In some embodiments, a blocking probe of the one or more blocking probes includes one or more abasic sites and the endonuclease is endonuclease IV. In some embodiments, a blocking probe of the one or more blocking probes includes a uracil and the enzyme is a uracil-specific excision reagent (USER).

In some embodiments, the blocking probe includes a poly(U) sequence, one or more RNA bases, one or more LNA bases, and combinations thereof. In some embodiments, a blocking probe of the one or more blocking probes, when hybridized to the analyte capture sequence or the capture domain, includes one or more mismatched nucleotides.

In some embodiments, the blocking probe has a length of about 8 to about 24 nucleotides. In some embodiments, the capture domain includes a nucleotide sequence of about 10 to 25 nucleotides in length. In some embodiments, the capture domain includes a unique nucleotide sequence.

In some embodiments, the analyte binding moiety is an antibody or an antigen-binding fragment thereof. In some embodiments, the analyte capture agent includes a linker, where the linker is disposed between the analyte binding moiety and the analyte binding moiety barcode. In some embodiments, the linker is a cleavable linker, and optionally, where the cleavable linker is a photo-cleavable linker or an enzyme cleavable linker.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic diagram of an exemplary analyte capture agent.

FIG. 3 shows an exemplary fluorescent image of a mouse spleen tissue where the analyte capture agents are inefficiently blocked.

FIG. 4 shows examples of capture probe domains of various lengths, 22nt (left) and 18, 16, 14 and 12 nt (right), aligned with associated analyte capture agent oligonucleotide sequences and experimental blocker configurations.

FIGS. 5A and B shows exemplary mouse spleen images of the effects of blocking the analyte capture agent oligonucleotide; A) background blocking and B) associated gene expression data; 14nt analyte capture agent oligonucleotide with blocking sequence TTGCTAGGA.

FIG. 6 shows exemplary antibody raw experimental data percentages for analyte capture agent oligonucleotide capture sequence lengths in combination with various blocker configuration as described in FIG. 4 on mouse spleen. Control was TotalSeqA antibody with 25nt polyT capture oligonucleotide (bottom row).

FIG. 7 shows exemplary gene expression raw experimental data percentages for experiments as found in FIG. 4.

FIG. 8 shows examples of capture probe domains of various lengths, 22nt (left) and 16 and 14nt (right), aligned with associated analyte capture agent oligonucleotide sequences and experimental blocker configurations.

FIG. 9 is an exemplary workflow for taking a tissue sample and performing the analyte capture including blocking as described herein.

FIG. 10 shows exemplary mouse spleen images of the effects of blocking the analyte capture agent oligonucleotide; A) background blocking and B) associated gene expression data; 16nt analyte capture agent oligonucleotide with blocking sequence TTGCTAIGACCIGCCT.

FIG. 11 shows exemplary gene expression raw experimental data percentages for analyte capture agent oligonucleotide capture lengths in combination with various blocker configurations as described in FIG. 9 on mouse spleen. Control data not shown.

FIG. 12 shows an example of a capture probe domain of 16nt length, aligned with an associated analyte capture agent oligonucleotide sequence and exemplary LNA blocker configuration.

FIGS. 13A and 13B show images of a human spleen tissue, wherein the tissue slides were processed and stained with unblocked antibodies (FIG. 13A) or antibodies blocked with the LNA blocker of FIG. 12 (FIG. 13B), and then imaged.

FIGS. 14A and 14B show images of UMI plots overlaid on the tissue image, where the capture domain is 16 nucleotides long (x16) and the analyte capture agent oligonucleotide is unblocked (FIG. 14A) or blocked with the LNA blocker probe of FIG. 12 (FIG. 14B).

### DETAILED DESCRIPTION

### I. Introduction

Disclosed herein are methods and kits for spatially determining the location of analytes within a biological sample. Determining the spatial location of analytes (e.g., protein) within a biological sample leads to better understanding of spatial heterogeneity in various contexts, such as disease models. In some embodiments of the methods for enhancing the specificity of binding an analyte capture sequence to a capture domain, an analyte capture agent includes an analyte binding moiety, an analyte capture sequence, and an analyte binding moiety barcode. In some embodiments, the analyte capture sequence is a nucleotide sequence. In some embodiments, the analyte capture sequence binds to a capture domain of a capture probe, where the capture probe includes a spatial barcode. In some embodiments, an array includes a plurality of capture probes, where a capture probe includes a spatial barcode and a capture domain.

Presently, improved methods of enhancing the specificity of binding an analyte capture sequence to a capture domain are needed. For example, the nucleotide sequence of an analyte capture sequence can non-specifically bind to a capture domain not covered by the biology sample. Non-specific binding of analyte capture sequences to capture domains results in skewed data and increased costs, such as sequencing. Thus, reversibly blocking (e.g., blocking with a blocking probe) the analyte capture sequence, the capture domain, or both enhance the specificity of binding the analyte capture sequence to the capture domain. In some embodiments, the analyte capture sequence, the capture domain, or both are blocked with one or more blocking probes. In some embodiments, the analyte capture sequence, the capture domain, or both are reversibly blocked during staining of the biological sample. In some embodiments, the blocking probes are released from the analyte capture sequence, the captured domain, or both. In some embodiments, the blocking probes are released from the analyte capture sequence, the capture domain, or both after staining the biological sample. In some embodiments, the blocking probes are released from the analyte capture sequence, the capture domain, or both after washing the biological sample. In some embodiments, the biological sample is permeabilized.

Thus, provided herein are methods for enhancing the specificity of binding of an analyte capture sequence of an analyte capture agent to a capture probe on an array (e.g., spatial array) by blocking the non-specific interactions between the analyte capture sequence and the capture probe.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10x Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or 0-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

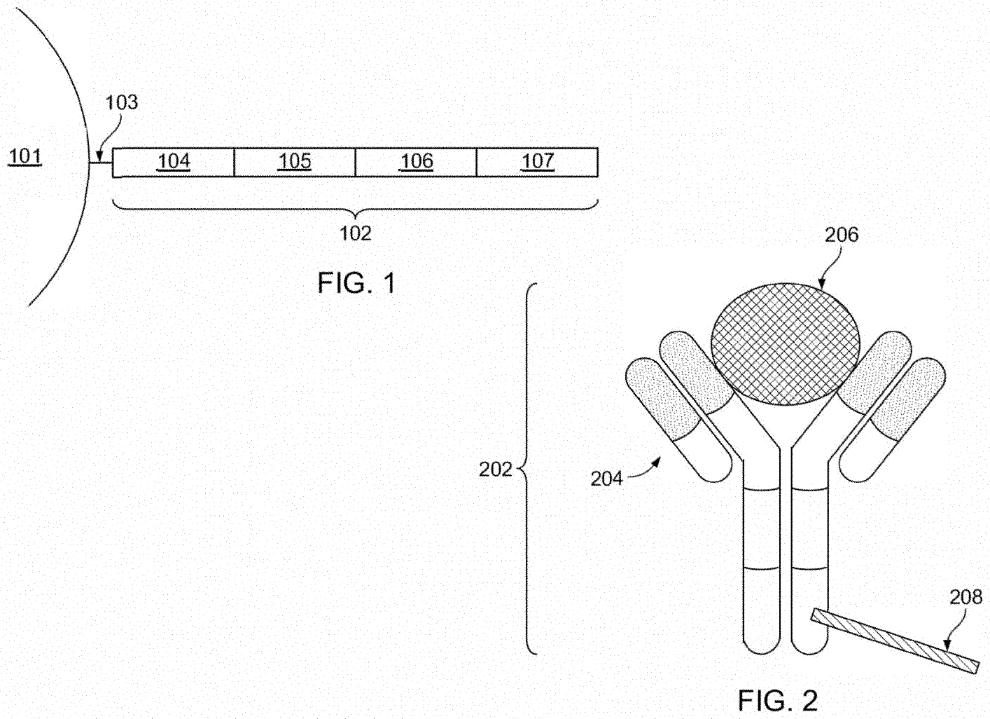

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Methods and Kits for Enhancing the Specificity of Analyte Capture Sequence Binding to Capture Domain

The present disclosure features methods and kits for spatially determining the location of analytes within a biological sample. Determining the spatial location of analytes (e.g., protein) within a biological sample leads to better understanding of spatial heterogeneity in various contexts, such as disease models. The methods and kits disclosed herein provide for enhancing the specificity of binding an analyte capture sequence to a capture domain. In some examples, an analyte capture agent includes an analyte binding moiety, an analyte capture sequence, and an analyte binding moiety barcode. In some examples, a linker is disposed between the analyte binding moiety and the analyte binding moiety barcode. In some examples, the analyte capture sequence is a nucleotide sequence. In some examples, the analyte binding moiety barcode is a nucleotide sequence. In some embodiments, the analyte binding moiety barcode identifies the analyte binding moiety. In some embodiments, the analyte capture sequence binds to a capture domain of a capture probe, where the capture probe includes a spatial barcode.

More specifically, the methods provided herein utilize blocking probes to block non-specific hybridization of an analyte capture sequence and the capture domain of a capture probe on an array thus enhancing the specificity of binding of an analyte capture sequence and a capture domain. In some examples, the blocking probes can differ in length and/or complexity. In some examples, the blocking probes specifically bind the analyte capture sequence. In some examples, the blocking probes specifically bind the capture domain. In some examples, the blocking probes specifically bind both the analyte capture sequence and the capture domain. In some examples, more than one blocking probe specifically binds the analyte capture sequence. In some examples, the blocking probe include one or more inosine nucleotides. In some examples, the blocking probes include one or more uracil nucleotides. In some examples, the blocking probes include one or more abasic sites. In some examples, the blocking probes are released by one or more of heat, cleavage, or washes in a salt buffer.

Provided herein are methods for binding an analyte capture sequence to a capture domain including: (a) contacting a biological sample with an array, where the array includes a plurality of capture probes including (i) a spatial barcode and (ii) a capture domain; (b) providing a plurality of analyte capture agents, where an analyte capture agent includes an analyte binding moiety that binds to an analyte in the biological sample, an analyte binding moiety barcode, and an analyte capture sequence, where the capture domain, the analyte capture sequence, or both are reversibly blocked with one or more blocking probes; and (c) releasing the one or more blocking probes from the capture domain, the analyte capture sequence, or both, and allowing the analyte capture sequence to specifically bind to the capture domain, thereby binding the analyte capture sequence to the capture domain in the biological sample.

In some embodiments, the blocking enhances the specificity of binding an analyte capture sequence to a capture domain compared to analyte binding specificity without blocking the capture domain, the analyte domain, or both.

In some embodiments, arrays are generated by attaching oligonucleotides together on a substrate surface. For example, an acceptor oligonucleotide can be attached to a functionalized substrate surface and a donor oligonucleotide can be attached (e.g., ligated) to the acceptor oligonucleotide on the surface of the substrate. In some embodiments, the acceptor oligonucleotide includes a cleavage domain, one or more functional domains, a unique molecular identifiers, and any combination thereof. In some embodiments the donor oligonucleotide is attached to the acceptor oligonucleotide by ligation. In some embodiments, the ligation reaction is facilitated by a splint oligonucleotide. For example, a splint oligonucleotide and be substantially complementary to a portion of the acceptor oligonucleotide and a portion of the donor oligonucleotide, such that the splint oligonucleotide hybridizes to both the acceptor oligonucleotide and the donor oligonucleotide and facilitates the ligation of donor oligonucleotide to the acceptor oligonucleotide to generate a capture probe.

In some embodiments, a donor oligonucleotide including the capture domain is ligated to an acceptor oligonucleotide on the surface of a substrate. In some embodiments, more than one (e.g., 2, 3, 4, or more) different types of donor oligonucleotides including different capture domains (e.g., different sequences (e.g., poly(T) vs a unique sequence), different lengths) are ligated to acceptor oligonucleotides on the surface of the substrate.

(a) Analyte Capture Agents

As described herein an “analyte capture agent” includes an analyte binding moiety (e.g., an antibody or antigen-binding fragment) and an analyte binding moiety barcode and an analyte capture sequence. In some embodiments, the analyte binding moiety is an antibody. In some embodiments, the analyte binding moiety is an antigen-binding fragment. In some embodiments, an analyte capture agent includes an analyte binding moiety and a capture agent barcode domain, wherein the capture agent barcode domain includes an analyte binding moiety barcode and an analyte capture sequence. In some embodiments, the analyte binding moiety barcode identifies the analyte binding moiety. In some embodiments, the analyte binding moiety is an antibody. In some embodiments, the antibody is a monoclonal antibody, a recombinant antibody, a synthetic antibody, a single domain antibody, a single-chain variable fragment (scFv), and or an antigen-binding fragment. In some embodiments, an analyte binding moiety can be an antibody or an antigen-binding fragment thereof, a cell surface receptor-binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. In some embodiments, the analyte binding moiety includes an antibody or antibody fragment that binds to an analyte (e.g., protein) in the biological sample. In some embodiments, the analyte is a protein. In some embodiments, the analyte binding moiety binds an analyte. In some embodiments, the analyte is a protein.

As used herein, the term “analyte capture sequence” refers to a region or moiety configured to hybridize to, bind to, couple to, or otherwise interact with a capture domain of a capture probe. In some embodiments, the analyte capture sequence is complementary or substantially complementary to the capture domain.

In some embodiments, the capture domain comprises SEQ ID NO: 1 (e.g., x12 Capture Domain). In some embodiments, the capture domain comprises SEQ ID NO: 2 (e.g., x14 Capture Domain). In some embodiments, the capture domain comprises SEQ ID NO: 3 (e.g., x16 Capture Domain). In some embodiments, the capture domain comprises SEQ ID NO: 4 (e.g., x18 Capture Domain). In some embodiments, the capture domain comprises SEQ ID NO: 5 (e.g., x22 Capture Domain).

In some embodiments, the analyte capture sequence is a homopolymeric sequence (e.g., poly(A) sequence). In some embodiments, the analyte capture sequence is a unique sequence (e.g., non-homopolymeric sequence). In some embodiments, the analyte capture sequence comprises SEQ ID NO: 6.

As used herein, the term “analyte binding moiety barcode” refers to a barcode that is associated with or otherwise identifies the analyte binding moiety. In some embodiments, by identifying an analyte binding moiety and its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein. For example, an analyte capture agent that is specific to one type of analyte can have coupled thereto a first capture agent barcode domain (e.g., that includes a first analyte binding moiety barcode), while an analyte capture agent that is specific to a different analyte can have a different (e.g., a second analyte binding moiety barcode) coupled thereto.

In some embodiments, the analyte capture agent includes a linker. In some embodiments, the linker is disposed between the analyte binding moiety and the analyte binding moiety barcode. In some embodiments, the linker is a cleavable linker. In some embodiments, the cleavable linker is a photo-cleavable linker. In some embodiments, the cleavable linker is a UV-cleavable linker. In some embodiments, the cleavable linker is an enzyme cleavable linker.

(b) Blocking Probes

The methods provided herein utilize blocking probes to block the non-specific binding (e.g., hybridization) of the analyte capture sequence and the capture domain of a capture probe on an array. In some embodiments, following contact between the biological sample and the array, the biological sample is contacted with a plurality of analyte capture agents, where an analyte capture agent includes an analyte capture sequence that is reversibly blocked with a blocking probe. In some embodiments, the analyte capture sequence is reversibly blocked with more than one blocking probe (e.g., 2, 3, 4, or more blocking probes).

The blocking probe can then be released from the analyte capture sequence, allowing the analyte capture sequence to specifically bind to the capture domain on the array. In some embodiments, blocking the analyte capture sequence reduces non-specific background staining. In some embodiments, the blocking probes are reversibly bound, such that the blocking probes can be removed from the analyte capture sequence during or after the time that analyte capture agents are in contact with the biological sample. In some embodiments, the blocking probe can be removed with RNAse treatment (e.g., RNAse H treatment). For example, if the blocking probe is an RNA blocking probe the blocking probe can be removed by RNase treatment. In some embodiments, the blocking probe includes one or more RNA bases and one or more DNA bases and is removed by RNase treatment. In some embodiments, the blocking probe includes one or more uracil nucleotides, one or more abasic sites, one or more mismatched nucleotides, one or more inosine nucleotides, one or more LNA bases, one or more RNA bases, one or more DNA bases, and any combination thereof and is removed by RNase treatment. In some embodiments, the blocking probes are removed by raising the temperature (e.g., heating) the biological sample. In some embodiments, the blocking probes are removed enzymatically (e.g., cleaved). In some embodiments, the blocking probes are removed by a USER enzyme. In some embodiments, the blocking probes are removed by an endonuclease. In some embodiments, the endonuclease is endonuclease IV. In some embodiments, the endonuclease is endonuclease V.

In some embodiments, blocking probes are hybridized to the analyte capture sequence of the analyte capture agents before introducing the analyte capture agents to a biological sample. In some embodiments, blocking probes are hybridized to the analyte capture sequence of the analyte capture agents after introducing the analyte capture agents to the biological sample. In such embodiments, the capture domain can also be blocked to prevent non-specific binding between the analyte capture sequence and the capture domain. In some embodiments, the blocking probes can be alternatively or additionally introduced during staining (e.g., immunofluorescent staining) of the biological sample. In some embodiments, the analyte capture sequence is blocked prior to binding to the capture domain, where the blocking probe includes a sequence complementary or substantially complementary to the analyte capture sequence.

In some embodiments, the analyte capture sequence is blocked with one blocking probe. In some embodiments, the analyte capture sequence is blocked with two blocking probes. In some embodiments, the analyte capture sequence is blocked with more than two blocking probes (e.g., 3, 4, 5, or more blocking probes). In some embodiments, a blocking probe is used to block the free 3′ end of the analyte capture sequence. In some embodiments, a blocking probe is used to block the 5′ end of the analyte capture sequence. In some embodiments, two blocking probes are used to block both 5′ and 3′ ends of the analyte capture sequence. In some embodiments, both the analyte capture sequence and the capture probe domain are blocked.

In some embodiments, the blocking probes can differ in length and/or complexity. In some embodiments, the blocking probe can include a nucleotide sequence of about 8 to about 24 nucleotides in length (e.g., about 8 to about 22, about 8 to about 20, about 8 to about 18, about 8 to about 16, about 8 to about 14, about 8 to about 12, about 8 to about 10, about 10 to about 24, about 10 to about 22, about 10 to about 20, about 10 to about 18, about 10 to about 16, about 10 to about 14, about 10 to about 12, about 12 to about 24, about 12 to about 22, about 12 to about 20, about 12 to about 18, about 12 to about 16, about 12 to about 14, about 14 to about 24, about 14 to about 22, about 14 to about 20, about 14 to about 18, about 14 to about 16, about 16 to about 24, about 16 to about 22, about 16 to about 20, about 16 to about 18, about 18 to about 24, about 18 to about 22, about 18 to about 20, about 20 to about 24, about 20 to about 22, or about 22 to about 24 nucleotides in length).

In some embodiments, the blocking probe includes one or more uracil nucleotides. In some embodiments, the blocking probe includes one or more abasic sites. In some embodiments, the blocking probe includes one or more mismatched nucleotides. For example, the one or more abasic sites can include Int 1′, 2′-dideoxyribose (dSpacer) (IDT Product 1202) that can generate one or more mismatched base pairings. In some embodiments, the blocking probe includes one or more inosine nucleotides. In some embodiments, the blocking probe includes one or more locked nucleic acids (LNA). In some embodiments, the blocking probe includes one or more RNA bases. In some embodiments, the blocking probe includes one or more DNA bases. In some embodiments, the blocking probe includes one or more RNA bases and one or more DNA bases (e.g., a combination of RNA and DNA bases). In some embodiments, the blocking probe includes one or more LNA bases and one or more RNA bases, DNA bases, or both. In some embodiments, the blocking probe includes one or more uracil nucleotides, one or more abasic sites, one or more mismatched nucleotides, one or more inosine nucleotides, one or more LNA bases, one or more RNA bases, one or more DNA bases, and any combination thereof.

In some embodiments, the buffer including the blocking probes includes an RNase. In some embodiments, the RNase is RNase I. In some embodiments, the buffer including the blocking probes includes a ribonucleoside vanadyl complex (RVC). In some embodiments, the buffer including the blocking buffer includes RVC and an RNase (e.g., RNase I). In some embodiments, the RNase is RNase H. In some embodiments, the RNase H is in an RNase H buffer.

In some embodiments, the blocking probe comprises SEQ ID NO: 7 (e.g., x8 Blocking Probe (3′)). In some embodiments, the blocking probe comprises SEQ ID NO: 8 (e.g., x9 Blocking Probe (3′)). In some embodiments, the blocking probe comprises SEQ ID NO: 9 (e.g., x9 Blocking Probe (5′)). In some embodiments, the blocking probe comprises SEQ ID NO: 10 (e.g., x8 Blocking Probe (5′)). In some embodiments, the blocking probe comprises SEQ ID NO: 11 (e.g., x12 USER Blocking Probe with Uracil). In some embodiments, the blocking probe comprises SEQ ID NO: 12 (e.g., x16 Inosine Blocking Probe). In some embodiments, the blocking probe comprises SEQ ID NO: 13 (e.g., x22 Inosine Blocking Probe). In some embodiments, the blocking probe comprises SEQ ID NO: 14 (e.g., x16 Abasic Blocking Probe). In some embodiments, the blocking probe comprises SEQ ID NO: 15 (e.g., x22 Abasic Blocking Probe). In some embodiments, the blocking probe comprises SEQ ID NO: 16 (e.g., x16 USER Blocking Probe with Uracil). In some embodiments, the blocking probe comprises SEQ ID NO: 17 (e.g., x22 USER Blocking Probe with Uracil). In some embodiments, the blocking probe comprises SEQ ID NO: 18 (e.g., Blocking Probe for x14 and x16 Capture Domain). In some embodiments, the blocking probe comprises SEQ ID NO: 19 (e.g., x14 USER Blocking Probe with Uracil). In some embodiments, the blocking probe comprises SEQ ID NO: 20 (e.g., x22 USER Blocking Probe with Uracil). In some embodiments, the blocking probe comprises SEQ ID NO: 22 (e.g., Capture Sequence 1 rBlock). In some embodiments, the blocking probe comprises SEQ ID NO: 23 (e.g., Capture Sequence 1 rBlock+_3). In some embodiments, the blocking probe comprises SEQ ID NO: 24 (e.g., Capture Sequence 1 rBlock+_5). In some embodiments, the blocking probe comprises SEQ ID NO: 25 (e.g., Capture Sequence 1 rBlock+_7). In some embodiments, the blocking probe comprises SEQ ID NO: 26 (e.g., LNA Blocker). In some embodiments, the blocking probe (e.g., blocking probes comprising SEQ ID NOs: 22-26) include an inverted 3′ base. In some embodiments, the inverted 3′ base is an inverted thymine base.

In some embodiments, one or more blocking probes are released by increasing the temperature of the biological sample when the one or more blocking probes are specifically bound (e.g., hybridized) to the analyte capture sequence or the capture domain and includes one or more mismatched nucleotides. In some embodiments, the one or more mismatched nucleotides in the blocking probe hybridized to the analyte capture sequence or the capture domain are positioned, after the fourth nucleotide from a 5′ end of the blocking probe and before the last four nucleotides at the 3′ end of the blocking probe.

In some embodiments, the one or more mismatched nucleotides in the blocking probe hybridized to the analyte capture sequence or the capture domain are positioned, after the sixth nucleotide from the 5′ end of the blocking probe and before the last six nucleotides at the 3′ end of the blocking probe.

In some embodiments, the capture domain is blocked prior to contacting the biological sample with the array. In some embodiments, the blocking probe is used to block the free 3′ end of the capture domain. In some embodiments, the blocking probe can be hybridized to the capture probe to mask the free 3′ end of the capture domain, e.g., hairpin probes, partially double stranded probes, or complementary sequences. In some embodiments, the blocking probe comprises SEQ ID NO: 21 (e.g., Capture Domain Blocking Probe (x9 slide)).

In some embodiments, the capture domain includes a nucleotide sequence of about 10 to about 25 (e.g., about 10 to about 20, about 10 to about 18, about 10 to about 16, about 10 to about 14, about 10 to about 12, about 12 to about 25, about 12 to about 20, about 12 to about 18, about 12 to about 16, about 12 to about 14, about 14 to about 25, about 14 to about 20, about 14 to about 18, about 14 to about 16, about 16 to about 25, about 16 to about 20, about 16 to about 18, about 18 to about 25, about 18 to about 20, or about 20 to about 25) nucleotides in length. In some embodiments, the capture domain includes a unique nucleotide sequence. In some embodiments, the capture domain is reversibly blocked with one blocking probe. In some embodiments, the capture domain is reversibly blocked with two blocking probes. In some embodiments, the capture domain is reversibly blocked with two or more blocking probes (e.g., 2, 3, 4, or more blocking probes).

(c) Analyte Capture Conditions

In some embodiments, the biological sample is fixed and stained prior to contacting the biological sample with the plurality of analyte capture agents. In some embodiments, the biological sample is fixed with an alcohol. In some embodiments, the alcohol is methanol. In some embodiments, the alcohol is 95%, 96%, 97%, 98%, 99%, or 100%. In some embodiments, the biological sample is fixed with an alcohol for about 15 minutes to about 50 minutes, for about 20 minutes to about 45 minutes, for about 25 minutes to 40 minutes, or for about 30 minutes to about 35 minutes. In some embodiments, the biological sample is fixed at about −5° C. to about −30° C., at about −10° C. to about −25° C., or at about −15° C. to about −20° C. In some embodiment, the biological sample is fixed in 100% methanol for 30 minutes at about −20° C.

In some embodiments, the biological sample is stained. In some embodiments, the biological sample is stained by immunofluorescence staining. In some embodiments, the biological sample is stained in a buffer. For example, SSC buffer, PBS, or TBS. In some embodiments, the biological sample is stained in about 1× saline sodium citrate (SSC) buffer to about 5×SSC buffer or about 2×SSC buffer to about 4×SSC buffer. In some embodiments, the biological sample is stained in about 3×SSC buffer. In some embodiments, the biological sample is stained for about 15 minutes to about 50 minutes, for about 20 minutes to about 45 minutes, for about 25 minutes to 40 minutes, or for about 30 minutes to about 35 minutes. In some embodiments, the biological sample is stained at about 0° C. to about 10° C., about 2° C. to about 8° C., or about 4° C. to about 6° C. In some embodiments, the biological sample is stained in 3×SSC for 30 minutes at 4° C.

In some embodiments, staining (e.g., staining under any of the conditions described herein) of the biological sample includes contacting the biological sample with a plurality of blocking probes, where a blocking probe of the one or more blocking probes specifically binds (e.g., hybridizes) to the capture domain, the analyte capture sequence, or both.

In some embodiments, the method includes washing the biological sample. For example, the biological sample can be washed 2, 3, 4, 5, or more times. In some embodiments, the washing includes a low salt wash buffer. In some embodiments, the low salt wash buffer is SSC buffer from about 0.01×SSC buffer to about 0.5×SSC buffer, 0.05×SSC buffer to about 0.3×SSC buffer, or about 0.1×SSC buffer to about 0.2×SSC buffer. In some embodiments, the low salt wash buffer is 0.1×SSC.

In some embodiments, the biological sample is washed to release the blocking probes from the capture domain, analyte capture sequence, or both. In some embodiments, releasing of the one or more blocking probes includes contacting the biological sample with an endonuclease enzyme. In some embodiments, the endonuclease enzyme is one or more of Endonuclease IV, Endonuclease V, or a uracil-specific excision reagent (USER) enzyme. In some embodiments, releasing the one or more blocking probes with an enzyme includes incubating for about 15 minutes to about 50 minutes, for about 20 minutes to about 45 minutes, for about 25 minutes to 40 minutes, or for about 30 minutes to about 35 minutes. In some embodiments, releasing the one or more blocking probes with an enzyme includes incubating for about 30 minutes. In some embodiments, incubating the blocking probes with an enzyme includes additional RVC and an RNase (e.g., RNase I). In some embodiments, incubating the blocking probes with an enzyme includes RNase H in an RNase H buffer.

In some embodiments, the biological sample is permeabilized. In some embodiments, the biological sample is permeabilized with a protease. In some embodiments, the protease is Proteinase K. In some embodiments, the protease is pepsin. In some embodiments, the biological sample is permeabilized with a detergent. In some embodiments, the detergent is Tween (e.g., Tween-20). In some embodiments, the detergent is TritonX 100. In some embodiments, the detergent is SDS. In some embodiments, the detergent (e.g., Tween, TritonX 100, or SDS) is present at a concentration of about 0.5% to about 2% or about 1% to about 1.5%. In some embodiments, the biological sample is permeabilized with a protease and a detergent. In some embodiments, the biological sample is permeabilized with Proteinase K and 1% SDS.

In some embodiments, the buffer, including the blocking probes, includes a protein, sera, or sera components that prevent non-specific antibody binding. For example, bovine serum albumin (BSA), human serum albumin (HAS), sera, or other serum components can be included in the buffer to reduce non-specific antibody binding.

In some embodiments, the analyte is a protein. In some embodiments, the protein is an intracellular protein. In some embodiments, the protein is an extracellular protein.

In some embodiments, the biological sample is a tissue sample. In some embodiments, the biological sample is a tissue section. In some embodiments, the tissue sample is a fixed tissue sample. In some embodiments, the tissue sample is a fixed tissue section. In some embodiments, the fixed tissue sample includes a formalin-fixed paraffin-embedded (FFPE) tissue sample. In some embodiments, the tissue sample is a fresh, frozen tissue section.

In some embodiments, all or a part of the sequence of the analyte binding moiety barcode, or a complement thereof, and all or a part of the sequence of the spatial barcode, or a complement thereof, is determined and the determined sequences are used to identify a location of the analyte in the biological sample. In some embodiments, determining all or a part of the sequence of the analyte binding moiety barcode or a complement thereof, and all or a part of the sequence of the spatial barcode or a complement thereof includes sequencing. In some embodiments, sequencing is high throughput sequencing.

(d) Kits

Also provided herein are kits including (a) an array, where the array comprises a plurality of capture probes, where a capture probe of the plurality of capture probes includes a spatial barcode and a capture domain and (b) a plurality of analyte capture agents, where an analyte capture agent of the plurality of analyte capture agents includes an analyte binding moiety that binds specifically to an analyte in a biological sample, an analyte binding moiety barcode, and an analyte capture sequence, where the capture domain, the analyte capture sequence, or both are reversibly blocked with one or more blocking probes.

In some kits, the capture domain is reversibly blocked with a blocking probe (e.g., any of the blocking probes described herein) of the one or more blocking probes. In some kits, the analyte capture sequence is reversibly blocked with a blocking probe (e.g., any of the blocking probes described herein) of the one or more blocking probes.

In some kits, the capture domain is reversibly blocked with a first blocking probe of the one or more blocking probes and the analyte capture sequence is reversibly blocked with a second blocking probe of the one or more blocking probes.

In some kits, the kit includes an enzyme. In some kits, the enzyme is an endonuclease. In some kits, the endonuclease is endonuclease V. In some kits, the endonuclease is endonuclease IV. In some kits, a blocking probe (e.g., any of the blocking probes described herein) of the one or more blocking probes includes one or more inosine nucleotides and the endonuclease is endonuclease V. In some kits, a blocking probe of the one or more blocking probes includes one or more abasic sites and the endonuclease is endonuclease IV. In some kits, a blocking probe of the one or more blocking probes includes one or more LNA bases. In some kits, a blocking probe includes one or more RNA bases. In some kits, a blocking probe includes one or more RNA bases and one or more LNA bases.

In some kits, a blocking probe of the one or more blocking probes includes a uracil and the enzyme is a uracil-specific excision reagent (USER). In some kits, the blocking probe includes a poly(U) sequence.

In some kits, a blocking probe (e.g., any of the blocking probes described herein) of the one or more blocking probes, when hybridized to the analyte capture sequence or the capture domain, includes one or more mismatched nucleotides.

In some kits, one or more mismatched nucleotides in the blocking probe hybridized to the analyte capture sequence or the capture domain are positioned, after the fourth nucleotide from a 5′ end of the blocking probe and before the last four nucleotides at the 3′ end of the blocking probe.

In some kits, one or more mismatched nucleotides in the blocking probe hybridized to the analyte capture sequence or the capture domain are positioned, after the sixth nucleotide from the 5′ end of the blocking probe and before the last six nucleotides at the 3′ end of the blocking probe.

In some kits, the blocking probe (e.g., any of the blocking probes described herein) has a length of about 8 to about 24 nucleotides.

In some kits, the capture domain includes a nucleotide sequence of about 10 to 25 nucleotides in length. In some kits, the capture domain includes a unique nucleotide sequence.

In some kits, the analyte is a protein. In some kits, the protein is an intracellular protein. In some kits, the protein is an extracellular protein.

In some kits, the analyte binding moiety is an antibody or an antigen-binding fragment thereof.

In some kits, the analyte capture agent includes a linker, where the linker is disposed between the analyte binding moiety and the analyte binding moiety barcode. In some kits, the linker is a cleavable linker. In some kits, the cleavable linker is a photo-cleavable linker. In some kits, the cleavable linker is an enzyme-cleavable linker.

### EXAMPLES

### Example 1—Methods for Blocking Non-Specific Binding of an Analyte Capture Sequence to a Capture Domain

An exemplary analyte capture agent 202 as described herein is shown in FIG. 2 and includes an analyte binding moiety 204 (e.g., an antibody or antigen-binding fragment) that binds an analyte 206, an analyte binding moiety barcode, and an analyte capture sequence 208 (shown as one sequence). The analyte capture sequence hybridizes to a capture domain of a capture probe at any location on an array. In some cases, a linker is disposed between the analyte binding moiety 204 and the analyte binding moiety barcode and analyte capture sequence 208. FIG. 1 is a schematic diagram showing an example of a capture probe, as described herein. As shown, the capture probe 102 is optionally coupled to a feature 101 by a cleavage domain 103, such as a disulfide linker. The capture probe can include functional sequences that are useful for subsequent processing, such as functional sequence 104, which can include a sequencer specific flow cell attachment sequence, e.g., a P5 sequence, as well as functional sequence 106, which can include sequencing primer sequences, e.g., a R1 primer binding site. In some embodiments, sequence 104 is a P7 sequence and sequence 106 is a R2 primer binding site. A spatial barcode 105 can be included within the capture probe for use in barcoding the target analyte. The capture probe can also include a capture domain 107 to facilitate capture of a target analyte.

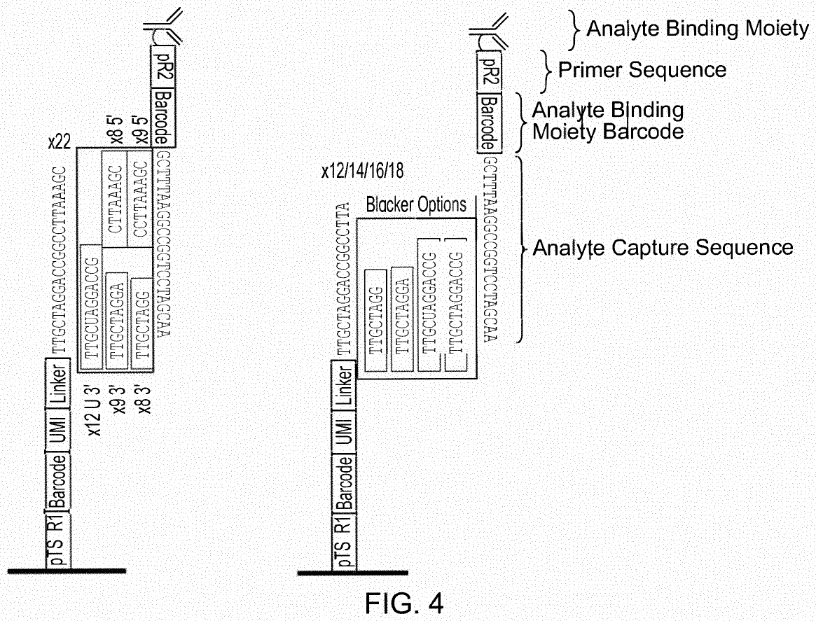

In some embodiments, as shown in FIG. 4, a capture probe can include a R1 primer binding site, a spatial barcode, a unique molecular identifier (UMI), a linker, and a capture domain. In some embodiments, the capture domain can include SEQ ID NO: 1 (e.g., x12 Capture Domain), SEQ ID NO: 2 (e.g., x14 Capture Domain), SEQ ID NO: 3 (e.g., x16 Capture Domain), SEQ ID NO: 4 (e.g., x18 Capture Domain), or SEQ ID NO: 5 (e.g., x22 Capture Domain.

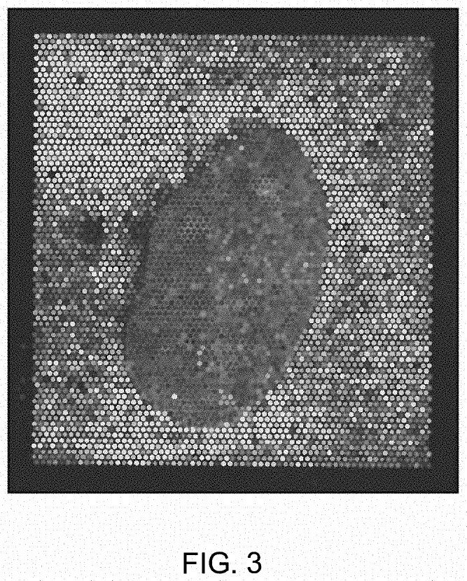

Background signal in spatial analysis results from a variety of factors. For example, analyte capture sequences present in an analyte capture agent can non-specifically bind to capture domains before the analyte binding moiety specifically binds its target analyte (e.g., a protein) and/or non-specifically binds capture domains outside the biological sample, thus leading to non-specific background signal as shown in FIG. 3. An exemplary method for blocking non-specific binding of an analyte capture sequence to a capture domain of a capture probe (e.g. enhancing specificity of binding of the analyte capture sequence to the capture probe) on an array includes utilizing a plurality of blocking probes, where a blocking probe is capable of reversibly blocking the capture domain, the analyte capture sequence, or both.

As shown in FIG. 4, an analyte capture agent incudes and analyte binding moiety (e.g., an antibody), an analyte binding moiety barcode, and an analyte capture sequence. Additionally, FIG. 4 shows exemplary blocking probe configurations for hybridizing the blocking probes to an analyte capture sequence of an analyte capture agent. The blocking probes can have different lengths and include one or more unique nucleotide sequences (uracil present in x12 U 3′), that hybridize to the analyte capture sequence. Also, included is a blocking probe configuration with two blocking probes hybridized to the analyte capture sequence (x9 3′ and x9 5′, x8 3′ and x8 5′).

Blocking hybridization of the analyte capture sequence and capture domain was tested with analyte capture sequences blocked with blocking probes of different lengths (e.g., 8, 9, or 12 nucleotides long) and capture domains of different lengths (e.g., 12, 14, 16, 18, or 22 nucleotides long). Blocking probes for the analyte capture sequence were incubated with the analyte capture agents in a staining mix for 30 minutes on ice. Following incubation the biological samples were washed in 3×SSC at room temperature. The x9 and x8 blocking probes biological sample experiments were rinsed in a low salt buffer (0.1×SSC) at 37° C. The USER samples were treated with USER in 1× Cutsmart Buffer at 37° C. for 30 minutes after imaging and rinsed with 3×SSC prior to permeabilization. Blocking probes were released from the analyte capture sequences prior to the biological sample being permeabilized, thus allowing the analyte capture sequence to hybridize to the capture domain of the capture probe.

Table 1 shows the blocking probe scheme used in this Example. The melting temperature (Tm) is based on 590 mM salt (Nat) in 3×SSC buffer and 20 μM of blocking probe. The Tm for uracil containing blocking probes is based on the longest fragment after cleavage.

TABLE 1
Blocking probe scheme

| | | Tm in 3x SSC | Tm in 0.1x SSC |
|---|---|---|---|
| Blocker Name | Blocker Sequence | (° C.) | (° C.) |
| x8 Blocker (3′) | TTGCTAGG | 41.4 | 22.4 |
| x9 Blocker (3′) | TTGCTAGGA | 44.4 | 27.1 |
| x9 Blocker (5′) | CCTTAAAGC | 43.8 | 23.7 |
| x8 Blocker (5′) | CTTAAAGC | 34.8 | 15 |
| x12 Blocker with U | TTGCUAGGACCG | 26.4 | 16.6 |

Immunofluorescence staining of nuclear material, CD-29 and CD-4 in mouse spleen samples fixed in 100% methanol demonstrated that the blocking probes did not affect the performance of the immunostaining or imaging where they were present during the immunofluorescence staining process.

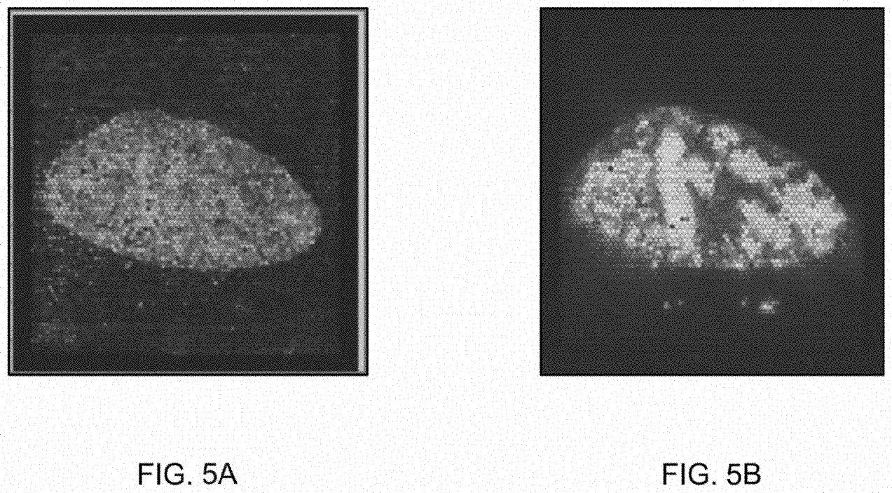

FIGS. 5A and B are representative images of one of the blocking configurations, where the capture domain is 14 nucleotides long (x14) and the blocking oligonucleotide to the analyte capture sequence is TTGCTAGGA (as depicted in FIG. 4, right side). FIG. 5A demonstrates that the blocking oligo for this configuration was able to greatly diminish background binding of the analyte capture sequence to the capture domains around the tissue. FIG. 5B is representative of gene expression data and that once the analyte capture sequence was deblocked (e.g., the block was removed), the analyte capture sequence was able to hybridize to the capture domain of the capture probe for downstream expression analysis of the target.

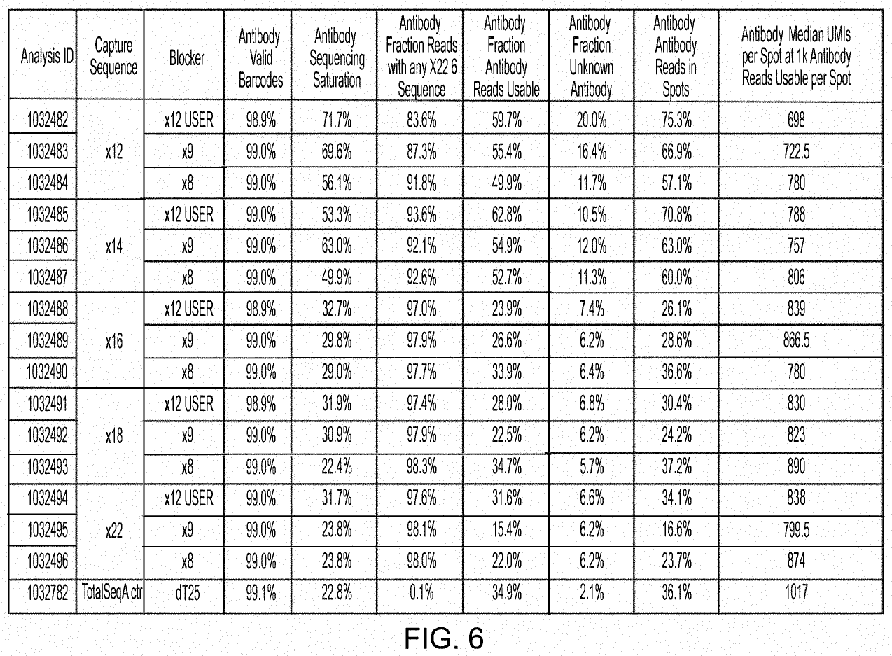

FIG. 6 summarizes the data for antibody reads showing the different capture domain lengths (e.g., capture sequence) and the blocking probes (e.g., blocker) used. In these experiments, the capture domain sequences resulted in the highest value of usable reads and antibody reads in spots, whereas the shorter capture sequences also showed decreased median UMIs per spot, and whereas with the longer capture sequences the data were generally reversed.

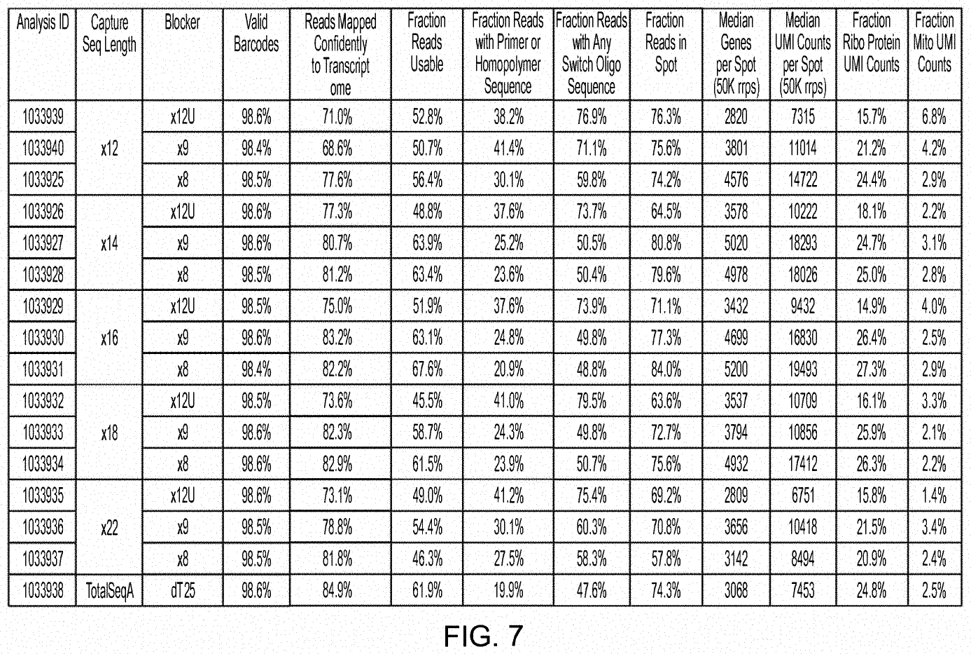

FIG. 7 summarizes with the spatial gene expression data, showing the different capture domains (e.g., capture sequence) and the blocking probes (e.g., blocker) used. In these experiments, gene expression data showed that the USER blocker decreased slightly the fraction of usable reads and mapped reads regardless of length. The mid-lengths of 14, 16, and 18 nucleotide capture sequences, using the shorter blockers (x9 and x8) had generally higher mapped and usable reads as well as median genes per spot and median UMI counts per spot.

The data demonstrate that shorter analyte capture sequences are easier to block relative to longer analyte capture sequences. For example, the data show that the blocking probes function better with x12 and x14 capture domain sequences. In contrast, x16, x18, and x22 capture domains have significantly lower fraction of unknown antibody.

The data also show that a higher fraction of antibody reads with a poly(A) sequence are captured relative to capture domains of 16, 18, or 22 nucleotides. Additionally, the USER blocking probe prevented nonspecific binding better than other blocking probes tested, but also resulted in reduced spatial gene expression data.

The data also demonstrated that the x14 capture domain in combination with the x9 blocking probe provided the best gene expression data in terms of mapping, usable reads, and sensitivity (e.g., ˜60% antibody reads per spot).

### Example 2—Methods for Blocking Non-Specific Binding of an Analyte Capture Sequence to a Capture Probe Using Different Blocking Probes

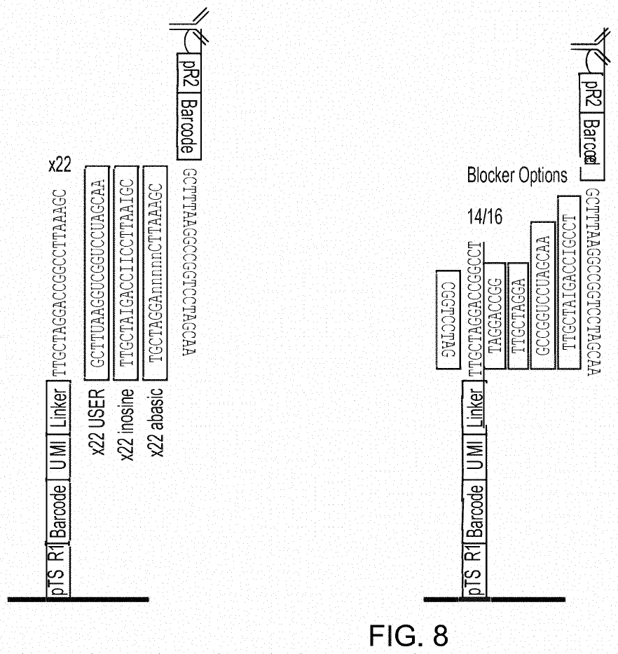

FIG. 8 shows an exemplary blocking scheme of hybridizing various blocking probes to an analyte capture sequence of an analyte capture agent and/or a capture domain (right) of a capture probe on an array. The blocking probes can have different lengths and include a unique nucleotide sequence, which allows specific binding of the blocking probe to the analyte capture sequence. Some blocking probes include one or more inosine nucleotides. Some blocking probes include one or more uracil nucleotides. Some blocking probes include one or more abasic sites.

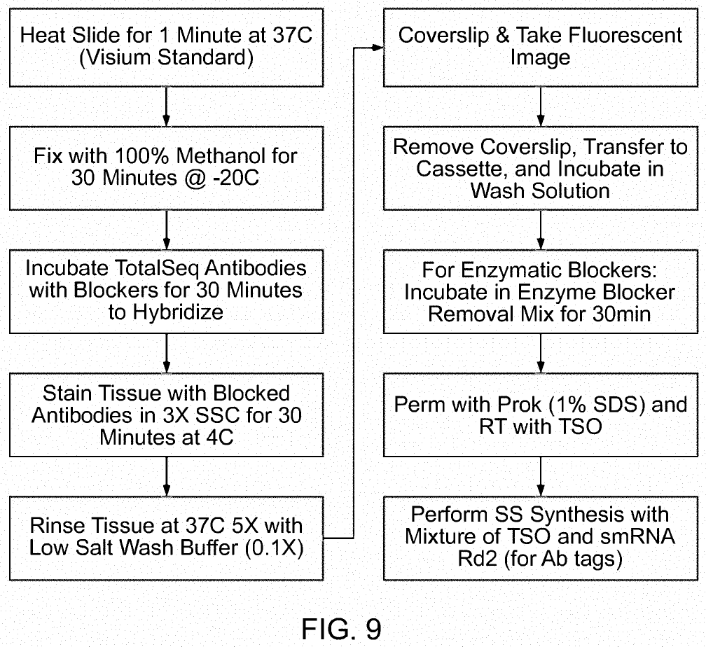

FIG. 9 is an exemplary spatial workflow for the detection of protein analytes in a biological sample. Blocking hybridization of the analyte capture sequence and the capture domain was tested with analyte capture sequences blocked with blocking probes of different lengths (e.g., 9, 14, 16, or 22 nucleotides long) and different compositions (e.g., inosine) and capture domains of different lengths (e.g., 14, 16, or 22 nucleotides long). The various blocking schemes tested are shown in in Table 2 below. The melting temperature (Tm) is based on 19.5 mM salt (Nat) in 0.1×SSC buffer and 20 μM of the blocking probe. The Tm for a uracil containing blocking probe, inosine blocking probe, and abasic blocking probe is based on the longest fragment after cleavage.

TABLE 2
Blocking Probe Schemes

| | | Tm in 3X | Tm in 0.1X |
|---|---|---|---|
| Blocker Name | Blocker Sequence | SSC (° C.) | SSC (° C.) |
| x9 Blocker (3′) | TTGCTAGGA | 47 | 27.1 |
| x9 Blocker 5′ | TAGGACCGG | 53.2 | 35.5 |
| x14/x16) | | | |
| x9 slide | CGGTCCTAG | 50.1 | 32.7 |
| x14 Blocker | GCCGGUCCUAGCAA | 72.2 | 18.9 |
| with U | | | |
| x16 Abasic | TTGCTAG/idSp//idSp//idSp/CGGCCT | 40 | 25.6 |
| x16 Inosine | TTGCTAIGACCIGCCT | 77.5 | 0.5 |
| x22 Abasic | TTGCTAGGA/idSp/ /idSp/ /idSp/ | 47 | 20.5 |
| | /idSp/ /idSp/CTTAAAGC | | |
| x22 Inosine | TTGCTAIGACCIICCTTAAIGC | 81.9 | 0 |
| x22 Blocker | GCTTUAAGGUCGGUCCUAGCAA | 76.8 | 0 |
| with U | | | |

Mouse spleen samples were fixed in 100% methanol for 30 minutes at −20° C. The TotalSeq antibodies (BioLegend) were incubated with the various blocking probes for 30 minutes to hybridize to the analyte capture sequence. The biological sample was stained and contacted with the analyte capture agents including the blocked analyte capture sequences in 3×SSC for 30 minutes at 4° C. After staining, the biological sample was rinsed in five times in 0.1×SSC at 37° C. Blocking probes removed via an enzyme were incubated in an enzyme blocker removal mix for the 30 minutes. For example, USER cleaves uracil, endonuclease V cleaves inosine, and endonuclease IV cleaves abasic sites. Blocking probes were released from the analyte capture sequences prior to the biological sample being permeabilized with Proteinase K and 1% SDS, thus allowing the analyte capture sequence to hybridize to the capture domain. Following capture of the analyte capture sequence by the capture domain, reverse transcription and second strand synthesis were performed followed by library construction and sequencing.

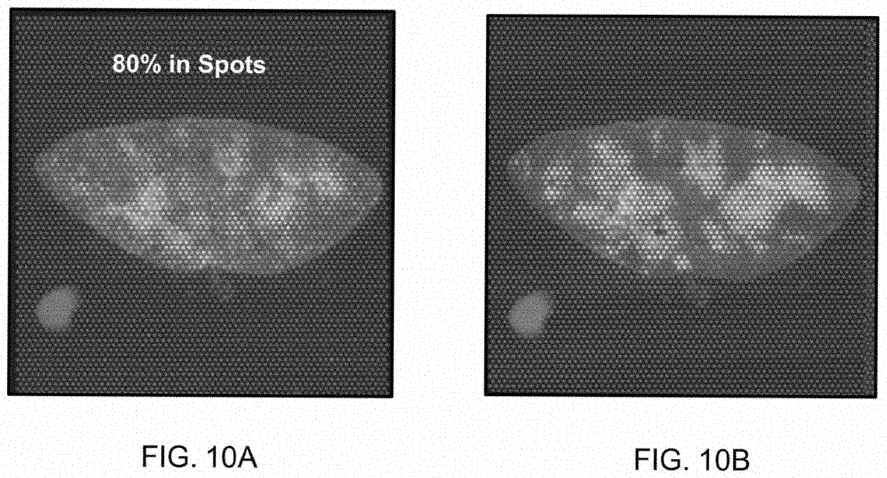

FIG. 10 are representative images showing the performance of the x16 inosine blocking probe against a capture domain of 16 nucleotides in length. The antibody signal is shown in A and spatial gene expression information is shown in B.

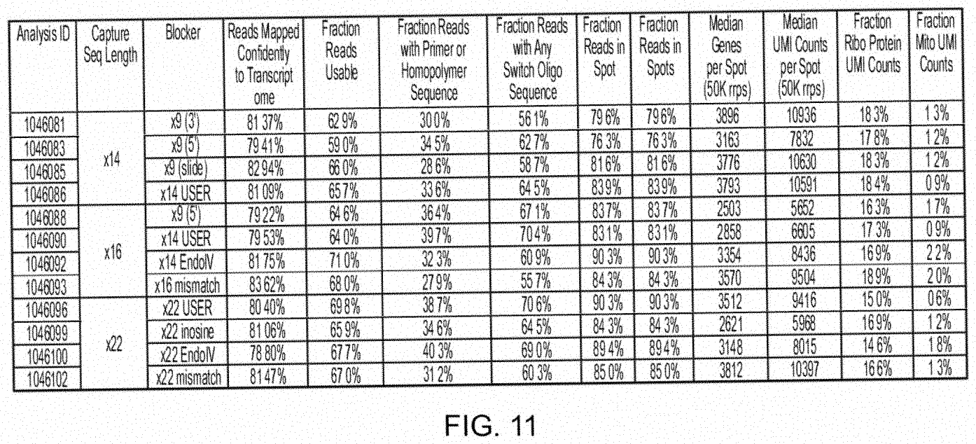

FIG. 11 summarizes the spatial gene expression data showing the different capture domains (e.g., capture sequence) and the blocking probes (e.g., blocker) used. The data demonstrate that high antibody usable reads and fractions of reads per spot are obtainable with proper blocking probe selection. The x16 and x22 capture domain sequences show a reduction in unknown antibody and lower fraction reads with poly(A) sequences relative to shorter capture domain sequences (e.g., x12 and x14). The spatial gene expression data also indicated a slight increase in template switching oligonucleotide and poly(A) sequences when USER enzyme was used and the x14 capture domain sequence showed the highest sensitivity, but the x16 capture domain sequence with a mismatch blocking probe demonstrated comparable sensitivity.

### Example 3—Methods for Tissue Optimization to Test Staining with Fluorescent Labelled Antibodies and Optimal Permeabilization Conditions

Antibody staining and tissue permeabilization are optimized by performing the methods or variations thereof disclosed herein. One example of a method of optimizing antibody staining and imaging can include: (a) providing a capture probe array, as described herein; (b) contacting the array with a tissue sample (˜10 μm tissue section) and drying the sectioned slides for 1 minute at 37° C.; (c) fixing the tissue sample with either 1% formaldehyde for 10 minutes at room temperature or with 100% methanol at −20° C. for 30 minutes or longer; (d) mounting slides into slide cassettes without drying the slides; (e) rehydrating and blocking the tissue sample; (f) removing the blocking buffer; (g) staining the tissue sample with fluorescent antibodies and blocking oligos in 3×SSC, 0.1% Tween, 2% BSA, and 2 U/μl RNAse inhibitor; (h) washing the tissue sample with; (i) dipping the tissue sample 3×SSC; (j) mounting tissue in mounting medium; and (k) imaging the tissue sample to evaluate the quality of fluorescent antibody staining.

Additionally, a method of optimizing permeabilization conditions for a biological sample can include: (a) providing an array, as described herein; (b) contacting the array with a tissue sample (e.g., −10 μm tissue section) and drying the sectioned slides for 1 minute at 37° C.; (c) fixing the tissue sample with either 1% formaldehyde for 10 minutes at room temperature or with 100% methanol at −20° C. for 30 minutes or longer; (d) mounting slides into slide cassettes without drying the slides; (e) rehydrating and blocking the tissue sample; (f) removing the blocking buffer; (g) staining the tissue sample with fluorescent antibodies and blocking oligos in 3×SSC, 0.1% Tween, 2% BSA, and 2 U/μl RNAse inhibitor for 30 minutes; (h) washing the tissue sample; (i) removing washing buffer from tissue sample; (j) incubating the tissue sample with a permeabilization mix of tissue removal enzyme, 3×SSC, and 10% SDS for 3, 6, 9, 12, 15, or 18 minutes at 37° C. to permeabilize the tissue and release the antibodies; (k) removing the permeabilization mix after the incubation period and washing twice with 0.1×SSC; and (1) performing a reverse transcription protocol and evaluating the optimal permeabilization conditions for different permeabilization times. Different samples of tissue can be treated with different permeabilization times (3, 6, 9, 12, 15, or 18 minutes) to identify the optimal permeabilization conditions for that particular sample type.

The protocol above describes antibody staining in 3×SSC buffer with 2% BSA and 0.2% Tween. However, it will be appreciated that other antibody staining buffer conditions or concentrations of these components may be more optimal for different antibodies and may be tested. For example, other components can include, but are not limited to, PBS or TBS based buffers, non-specific antibody binding blocking with other components than BSA, such as sera or serum components, and other detergents such as TritonX 100.

### Example 4—Methods of Library Preparation for Protein Detection

Library preparation for protein detection requires different buffers and reagents compared to standard Visium library preparation protocol. The following protocol was used after establishing optimal permeabilization and antibody staining conditions.

A 2x Blocking Buffer including SSC, Tween, BSA, sheared salmon sperm, and an RNase Inhibitor, can be prepared beforehand. Additionally, an Antibody Staining Mix comprising 1x Blocking Buffer, Blocking Oligos (dT25), RNAse Inhibitor, fluorescent antibodies, and Totalseq A Antibody (BioLegend) pool can be prepared beforehand as well as a Washing Buffer. Finally, the Mounting Medium for the slide can comprise 90% glycerol and an RNase Inhibitor. These buffers and reagents can be used in the methods below for protein/antibody detection in combination with the oligonucleotide workflows described herein.

In one example, a method for preparing a TotalSeqA (BioLegend) antibody panel can include: (a) pooling an appropriate amount of TotalSeqA antibodies to create a panel of interest; (b) preparing an Amicon Ultra-0.5 50 kDa MWCO filter unit with 3×SSC; (c) adding the antibody pool to the filter and spinning the unit at 14,000 g for 5 minutes; (d) discarding the flow through and adding 3×SSC; (e) spinning the sample at 14,000 g for 5 minutes; and (f) inverting the filter into a collection tube and spinning the collection tube at 1,000 g for 2 minutes, thereby recovering the antibody pool. In some embodiments, when pooling large numbers of antibodies, the storage buffer comprises 3×SSC. In some embodiments, 1 μg/μl BSA and 0.06% sodium azide is added to the recovered antibody pool.

In one example, a method of library preparation for protein detection can include: (a) providing a capture probe array, as described herein; (b) contacting the substrate with a tissue sample (e.g., ˜10 μm tissue section) and drying the sectioned slides for 1 minute at 37° C.; (c) fixing the tissue sample with either 1% formaldehyde for 10 minutes at room temperature or with 100% methanol at −20° C. for 30 minutes or longer; (d) mounting slides into slide cassettes without drying the slides; (e) rehydrating and blocking the tissue sample; (f) removing blocking buffer from the tissue; (g) staining the tissue sample with fluorescent antibodies and blocking oligonucleotides in SSC, Tween, BSA, and an RNase inhibitor; (h) washing the tissue sample in SSC wash solution; (i) dipping tissue slide in SSC; (j) imaging the tissue sample to detect visible antibodies (e.g., Cy3), wherein a fiducial frame is visible on the slide; (k) washing the tissue with washing buffer and removing the washing buffer; (1) incubating the tissue sample with an even covering of tissue removal enzyme, SSC, and SDS to permeabilize the tissue and release the antibodies for an optimal amount of time, as determined in Example 3; (m) removing the tissue from the permeabilization mix and washing twice with 0.1×SSC; and (n) performing a reverse transcription protocol according to methods described herein.

In another example, a method of library preparation for protein detection with second strand synthesis can include: (a) providing a capture probe array, as described herein; (b) contacting the substrate with a tissue sample (e.g., ˜10 μm tissue section) and drying the sectioned slides for 1 minute at 37° C.; (c) fixing the tissue sample with either 1% formaldehyde for 10 minutes at room temperature or with 100% methanol at −20° C. for 30 minutes or longer; (d) mounting slides into slide cassettes without drying the slides; (e) rehydrating and blocking the tissue sample; (f) removing blocking buffer from the tissue; (g) staining the tissue sample with fluorescent antibodies and blocking oligos; (h) washing the tissue sample; (i) dipping tissue slide in SSC; (j) imaging the tissue sample to detect visible antibodies, wherein a fiducial frame (e.g. Cy3) is visible on the slide; washing the tissue with washing buffer and removing the washing buffer; (1) treating the tissue sample with an even covering of tissue removal enzyme, SSC, and SDS to permeabilize the tissue and release the antibodies for an optimal amount of time, as determined in Example 3; (m) removing the tissue from the permeabilization mix and washing twice with 0.1×SSC; (n) performing a reverse transcription protocol; and (i) adding additive primer to a second strand synthesis mix and perform second strand synthesis according to the methods described herein. For example, second strand synthesis can be performed by removing a reverse transcriptase master mix from the tissue; adding KOH to the tissue; adding elution buffer to the tissue; removing elution buffer from the tissue; adding a second strand mix to the tissue, wherein the second strand mix comprises second strand reagent, second strand primer, second strand enzyme, and additive primers; subjecting the tissue to a thermocycling program comprising 65° C. for second strand synthesis followed by a 4° C. hold.

In another example, a method of cDNA amplification and cleanup can include: (a) preparing a cDNA amplification mix on ice; (b) adding an additive primer and cDNA primers to the cDNA amplification mix to increase yield of antibody-products; and (c) performing cDNA amplification as described herein. In some embodiments, additive primer is not added to qPCR to determine amplification cycles. In some embodiments, cDNA amplification is performed one additional cycle than what was determined by qPCR.

In another example, a method of cDNA and antibody-product size selection can include: (a) separating cDNA amplification antibody-products (e.g., ˜180 bp) and mRNA-derived cDNAs (e.g., >300 bp) by SPRI beads, wherein the bead fraction contains mRNA derived cDNAs and supernatant contains ADTs; (b) adding SPRI reagent to the cDNA reaction and incubating for 5 minutes at room temperature; (c) placing the cDNA reaction on the magnet high position for ˜1 minute until solution is clear; (d) transferring the supernatant to a low-bind tube; and (e) performing cDNA cleanup and library preparation with as described herein with beads. In some embodiments, the supernatant is transferred to a low-bind tube and used to perform antibody-product cleanup. Other library preparation steps can be completed as described herein.

In another example, a method of antibody-product cleanup can include: (a) purifying antibody-products from highly concentrated cDNA amplification primers by two rounds of SPRI cleanups; (b) adding SPRI beads to supernatant to obtain a final SPRI to sample ratio of 1.9× and incubating 5 minutes at room temperature; (c) placing the tube on a magnet until the solution is clear; (d) removing and discarding the supernatant; (e) adding 80% ethanol to the tube and removing the ethanol wash; (f) resuspending beads in water; (g) performing another round of SPRI purification by adding SPRI reagent directly onto resuspended beads and incubating 5 minutes at room temperature; (h) placing tube on magnet until solution is clear; (i) removing and discarding the supernatant; (j) adding 80% Ethanol, without disturbing the pellet, letting stand for 30 seconds and removing the ethanol wash; (k) repeating the ethanol wash; (1) air drying the beads and resuspending beads in water; and (m) placing tube on magnet and transferring clear supernatant into a PCR tube.

In another example, a method of antibody sequencing library amplification can include: (a) preparing a PCR reaction of purified ADTs, wherein the PCR reaction comprises purified antibody-product, amplification mix, TruSeq small RNA RPIx primer, and SI-PCR primer; (b) cycling the PCR reaction: 95° C. for 3 minutes, 95° C. for 20 seconds, 60° C. for 30 seconds, 72° C. for 20 seconds, 72° C. for 5 minutes, for approximately 6-10 cycles; (c) purifying antibody PCR product by adding SPRI reagent to sample and incubating 5 minutes at room temperature; (d) placing tube on magnet high position until solution is clear; (e) removing and discarding the supernatant; (f) adding 80% Ethanol to the tube for 30 seconds and removing the ethanol wash; (g) repeating the ethanol wash; (h) air drying the beads and resuspending beads in water; (i) mixing the beads and water and incubating at room temperature for 5 minutes; (j) placing tube on magnet and transferring clear supernatant into a PCR tube; (k) quantifying the prepared antibody libraries by standard methods described herein (antibody libraries can be ˜180 bp; and (1) sequencing the antibody libraries. Other library preparation steps can be completed as described herein.

### Example 5—Blocking Probes with LNA and RNA Bases

Additional types of blocking probes, including blocking probes with locked nucleic acid (LNA) bases and/or RNA bases, were also tested. For example, a blocking probe including SEQ ID NO: 22 contains RNA bases and blocking probes including SEQ ID NOs: 23-26 include both RNA bases and either 3, 5, or 7 LNA bases, respectively. Blocking probes, including blocking probes with RNA bases, were released by RNAse H in an RNAse H buffer, which specifically cleaves RNA in a DNA-RNA hybrid duplex. Releasing the blocking probe allows the analyte capture agent and, more specifically, the analyte capture sequence to specifically bind a capture domain of a capture probe.

qPCR data demonstrated that blocking an oligonucleotide (e.g., an oligonucleotide that mimicked an analyte capture sequence) with a blocking probe including one or more RNA bases (e.g., SEQ ID NOs: 22-26), followed by unblocking (e.g., releasing) with RNAse H treatment allowed the oligonucleotide to interact with a capture domain of a capture probe. For example, the affinity of an oligonucleotide to a capture domain of a capture probe was measured by qPCR. If the oligonucleotides were not blocked at all, the oligonucleotides were captured and detected by qPCR (cycle threshold (CT) ˜9). When the oligonucleotides were blocked with a blocking probe negligible amplification occurred (CT ˜20). If, however, the oligonucleotides were unblocked with RNase H treatment in RNAse H buffer, the negligible amplification was reversed and amplification occurred at similar levels to the oligonucleotides that were not blocked at all (CT ˜10) (qPCR data not shown).

### Example 6—Methods for Blocking Non-Specific Binding of an Analyte Capture Sequence to a Capture Probe Using LNA Blocking Probes

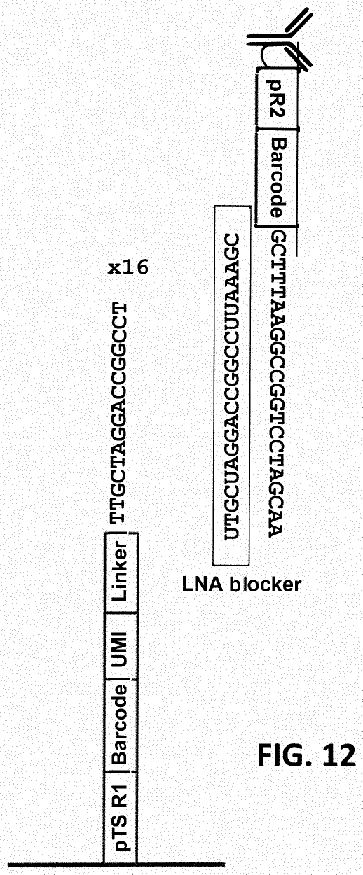

FIG. 12 shows an exemplary blocking scheme of hybridizing an LNA blocking probe to an analyte capture sequence of an analyte capture agent. The blocking probe can have a unique nucleotide sequence, which allows specific binding of the blocking probe to the analyte capture sequence. In some embodiments, an LNA blocking probe can include one or more LNA bases (e.g, SEQ ID NO: 26)

In one example, a method of using an LNA blocking probe (e.g., LNA blocker) against an analyte capture sequence of 16 nucleotides in length can include the steps described herein. Briefly, FFPE human spleen tissues were sectioned, mounted on spatial array slides and deparaffinized using a series of xylene (2×10 minute incubations) and ethanol washes (2×3 minute incubations in 100% ethanol) prior to drying at room temperature. The slides were heated at 37° C. for 15 minutes, followed by a series of 3 minute ethanol washes (100%, 96%, 96%, and 70% ethanol). The tissues were H&E stained and brightfield imaged. Alternatively, tissues can be stained (e.g., immunofluorescence stained) instead of H&E staining.

Tissues were washed and decrosslinked by incubating the tissues in Tris-EDTA (TE) buffer (pH 9.0) for 1 hour at 95° C. followed by a series (3) of 1 minute washes with 0.1 N HCl. After decrosslinking the tissues, the targeted RTL probes were added to the tissues and probe hybridization ran overnight at 50° C. The tissues were then washed in a post-hybridization buffer including 3×SSC, 7% ethylene carbonate, Baker's yeast tRNA, and nuclease free water) and followed by a 2×SSC buffer wash. Post-hybridization, the probes were ligated together at 37° C. for 1 hour. Following RTL probe hybridization, the tissues were incubated in an antibody blocking buffer (PBS-based buffer (pH 7.4), 5% goat serum, 0.1 μg/μL salmon sperm DNA, 0.1% Tween-20, an 1 U/μL RNase inhibitor, and 10 mg/mL dextran sulfate) with the tissues at room temperature for 60 minutes. The blocking buffer was removed from the tissues and (PBS-based buffer (pH 7.4), 5% goat serum, 0.1 μg/μL salmon sperm DNA, 0.1% Tween-20, an 1 U/μL RNase inhibitor, blocking oligonucleotides, analyte capture agents (e.g., antibodies with a conjugated oligonucleotide) and 10 mg/mL dextran sulfate) overnight at 4° C. The tissue samples were then washed four times with antibody staining buffer without antibodies.

Tissues were permeabilized and the ligated RTL probes were released for capture by hybridization to the capture domains of the capture probes on the spatial array surface. The oligonucleotides of the analyte capture agents complementary to the alternative capture sequences of the second set of capture probes on the array were also captured by hybridization. As such, both RTL ligation products representing the mRNA of the targeted protein and the oligonucleotide of the analyte capture agent representing the binding of the antibody to the targeted protein were concurrently captured on the array surface. To allow for probe and oligonucleotide release and capture, the tissues were incubated with an RNase (e.g., RNase Hand), an associated buffer, and polyethylene glycol (PEG) for 30 minutes at 37° C. Tissues were permeabilized using a permeabilization buffer comprising a protease (e.g., Proteinase K), PEG, 3M urea, for an additional 60 minutes, followed by washing to remove the enzymes from the tissues. After permeabilization the tissues were washed in 2×SSC three times.

The captured RTL ligation products and the analyte binding agent oligonucleotides were extended to create second strand cDNA products of the captured molecules including the spatial barcode, the analyte binding moiety barcode if present and other functional sequences from the capture probe. Additionally, said products were pre-amplified prior to library preparation.

Library preparations were made from the second strand cDNA products, the libraries sequenced on an Illumina sequencing instrument, and spatial locations determined using Space Ranger and Loupe Browser (10X Genomics). The antibody sequences (e.g., the complement of the captured oligonucleotide from the analyte binding agents) were amplified with Truseq_pR1 and Truseq_pR2. For protein localization, sequences relating to the analyte binding moiety barcode were used to determine abundance and location of the labeled protein by the analyte binding agents. Spatial expression patterns were determined using SpaceRanger data analysis software and Loupe browser visualization software (10X Genomics).

The FFPE human spleen tissues were stained with either unblocked antibodies or antibodies blocked with the LNA blocking (FIG. 12) probe overnight. Secondary staining was performed with Cy3-conjugated secondary antibodies and the tissue sample was imaged.

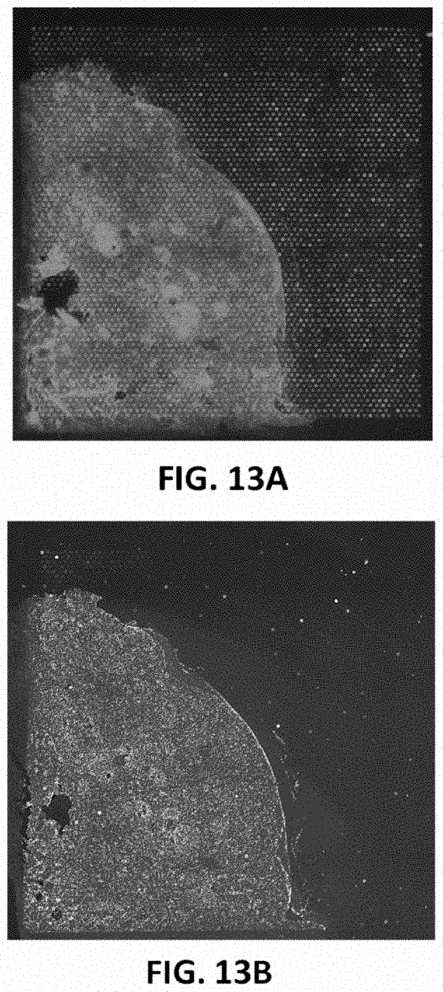

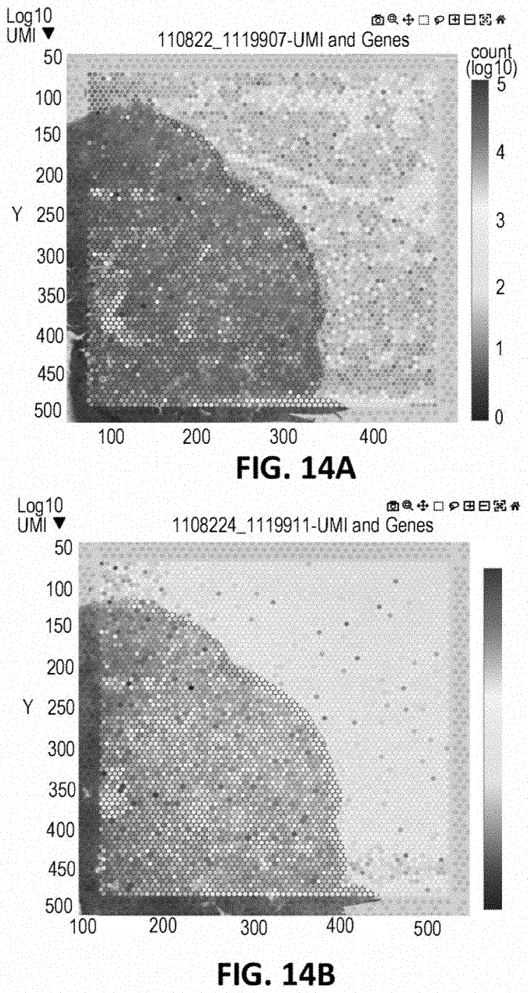

FIGS. 13A and 13B are images of an FFPE human spleen tissue, wherein the tissue samples were processed by the methods described above, secondary stained with Cy3 and imaged. The image where the antibody was unblocked (FIG. 13A) or blocked with a LNA blocker (FIG. 13B), shows a significant reduction in background staining around the tissue when the antibody is blocked FIG. 13B compared to unblocked FIG. 13A. FIGS. 14A and 14B are images of UMI plots, where the capture domain on the spatial array is 16 nucleotides long (x16) and the blocking oligonucleotide to the analyte capture sequence is an LNA blocker (FIG. 12). FIG. 14B demonstrates that an LNA blocking oligonucleotide was able to significantly diminish background binding of the analyte capture sequence to the capture domains around the tissue sample as compared to FIG. 14A where no LNA blocking oligonucleotide was used.

SEQ ID NO: APPENDIX

SEQ ID NO: 1 x12 Capture domain
TTGCTAGGACCG
SEQ ID NO: 2 x14 Capture domain
TTGCTAGGACCGGC
SEQ ID NO: 3 x16 Capture domain
TTGCTAGGACCGGCCT
SEQ ID NO: 4 x18 Capture domain
TTGCTAGGACCGGCCTTA
SEQ ID NO: 5 x22 Capture domain
TTGCTAGGACCGGCCTTAAAGC
SEQ ID NO: 6 Analyte Capture Sequence
GCTTTAAGGCCGGTCCTAGCAA
SEQ ID NO: 7 x8 Blocking Probe (3′)
TTGCTAGG
SEQ ID NO: 8 x9 Blocking Probe (3′)
TTGCTAGGA
SEQ ID NO: 9 x9 Blocking Probe (5′)
CCTTAAAGC
SEQ ID NO: 10 x8 Blocking Probe (5′)
CTTAAAGC
SEQ ID NO: 11 x12 USER Blocking Probe with Uracil (U)
TTGCUAGGACCG
SEQ ID NO: 12 x16 Inosine Blocking Probe
TTGCTAIGACCIGCCT
SEQ ID NO: 13 x22 Inosine Blocking Probe
TTGCTAIGACCIICCTTAAIGC
SEQ ID NO: 14 x16 Abasic Blocking Probe *
TTGCTAG/idSp//idSp//idSp/CGGCCT
SEQ ID NO: 15 x22 Abasic Blocking Probe*
TTGCTAGGA/idSp//idSp//idSp//idSp//idSp/CTTAAAGC
*SEQ ID NOs: 14 and 15: idSP = Int 1′,3′-Dideoxyribose (dSpacer)
SEQ ID NO: 16 x16 USER Blocking Probe with Uracil (U)
TTGCUAGGACUGGC
SEQ ID NO: 17 x22 USER Blocking Probe with Uracil (U)
TTGCUAGGACCUGCCUTAAAGC
SEQ ID NO: 18 x9 Blocking Probe for x14 and x16 Capture Domain
TAGGACCGG
SEQ IN NO: 19 x14 USER Blocking Probe with Uracil (U)
GCCGGUCCUAGCAA
SEQ IN NO: 20 x22 USER Blocking Probe with Uracil (U)
GCTTUAAGGUCGGUCCUAGCAA
SEQ ID NO: 21 Capture Domain Blocking Probe (x9 slide)
CGGTCCTAG
SEQ ID NO: 22 Capture Sequence 1 rBlock*
rUrUrGrCrUrArGrGrArCrCrGrGrCrCrUrUrArArArGrC/3InvdT/
SEQ ID NO: 22 Capture Sequence 1 rBlock+_3*
rU + TrGrCrUrArGrGrArCrC + GrGrCrCrUrUrArArA + GrC/3InvdT/
SEQ ID NO: 24 Capture Sequence 1 rBlock+_5*
rU + TrGrCrUrA + GrGrArCrC + GrGrCrC + TrUrArArA + GrC/3InvdT/
SEQ ID NO: 25 Capture Sequence 1 rBlock+_7*
rU + TrGrC + TrArG + GrArC + CrGrG + CrCrU + TrArA + ArGrC/3InvdT/
SEQ ID NO: 26 LNA Blocker
rU + TrGrCrUrArGrGrArCrC + GrGrCrCrUrUrArArA + GrC/3InvdT/
*SEQ ID NOs: 22-26: A “+” in front of a base indicates the base is a locked
nucleic acid (LNA) base, an “r” in front of a base indicates the base is an
RNA base, and 3InvdT is an inverted thymine base.

### Embodiments

Accordingly, the present disclosure provides:

Embodiment 1 is a method for binding an analyte capture sequence to a capture domain comprising: (a) contacting a biological sample with an array, wherein the array comprises a plurality of capture probes comprising (i) a spatial barcode and (ii) a capture domain; (b) providing a plurality of analyte capture agents, wherein an analyte capture agent comprises an analyte binding moiety that binds to an analyte in the biological sample, an analyte binding moiety barcode, and an analyte capture sequence, wherein the capture domain, the analyte capture sequence, or both are reversibly blocked with one or more blocking probes; and (c) releasing the one or more blocking probes from the capture domain, the analyte capture sequence, or both, and allowing the analyte capture sequence to specifically bind to the capture domain, thereby binding the analyte capture sequence to the capture domain in the biological sample.

Embodiment 2 is the method of embodiment 1, wherein the blocking enhances the specificity of binding an analyte capture sequence to a capture domain compared to analyte binding specificity without blocking the capture domain, the analyte domain, or both.

Embodiment 3 is the method of embodiment 1 or 2, further comprising, prior to step (b), fixing and staining the biological sample.

Embodiment 4 is the method of embodiment 3, wherein fixing comprises methanol.

Embodiment 5 is the method of embodiment 3 or 4, wherein the staining comprises immunofluorescence staining.

Embodiment 6 is the method of embodiment 1, wherein, prior to the contacting in step (b), contacting the plurality of analyte capture agents with the one or more blocking probes.

Embodiment 7 is the method of any one of embodiments 1-6, wherein the capture domain is reversibly blocked with a blocking probe of the one or more blocking probes.

Embodiment 8 is the method of any one of embodiments 1-6, wherein the analyte capture sequence is reversibly blocked with a blocking probe of the one or more blocking probes.

Embodiment 9 is the method of any one of embodiments 1-6, wherein the capture domain is reversibly blocked with a first blocking probe of the one or more blocking probes and the analyte capture sequence is reversibly blocked with a second blocking probe of the one or more blocking probes.

Embodiment 10 is the method of any one of embodiments 1-9, wherein the releasing of the one or more blocking probes comprises the use of an enzyme.

Embodiment 11 is the method of embodiment 10, wherein the enzyme is an endonuclease.

Embodiment 12 is the method of embodiment 11, wherein a blocking probe of the one or more blocking probes comprises one or more inosine nucleotides and the endonuclease is endonuclease V.

Embodiment 13 is the method embodiment 11, wherein a blocking probe of the one or more blocking probes comprises one or more abasic sites and the endonuclease is endonuclease IV.

Embodiment 14 is the method of embodiment 10, wherein a blocking probe of the one or more blocking probes comprises a uracil and the enzyme is a uracil-specific excision reagent (USER).

Embodiment 15 is the method of embodiment 14, wherein the blocking probe comprises a poly(U) sequence, one or more RNA bases, one or more LNA bases, and combinations thereof.

Embodiment 16 is the method of any one of embodiments 1-9, wherein a blocking probe of the one or more blocking probes, when hybridized to the analyte capture sequence or the capture domain, comprises one or more mismatched nucleotides, and the releasing comprises increasing the temperature of the biological sample.

Embodiment 17 is the method of embodiment 16, wherein one or more mismatched nucleotides in the blocking probe hybridized to the analyte capture sequence or the capture domain are positioned, after the fourth nucleotide from a 5′ end of the blocking probe and before the last four nucleotides at the 3′ end of the blocking probe.

Embodiment 18 is the method of embodiment 17, wherein one or more mismatched nucleotides in the blocking probe hybridized to the analyte capture sequence or the capture domain are positioned, after the sixth nucleotide from the 5′ end of the blocking probe and before the last six nucleotides at the 3′ end of the blocking probe.

Embodiment 19 is the method of any one of embodiments 12-18, wherein the blocking probe has a length of about 8 to about 24 nucleotides.

Embodiment 20 is the method of any one of embodiments 10-19, wherein the releasing of the one or more blocking probes further comprises washing the biological sample.

Embodiment 21 is the method of embodiment 20, wherein the washing comprises the use of a buffer comprising about 0.01× to about 0.5× saline sodium citrate (SSC).

Embodiment 22 is the method of any one of embodiments 1-21, wherein the method further comprises permeabilizing the biological sample.

Embodiment 23 is the method of any one of embodiments 1-22, wherein the capture domain comprises a nucleotide sequence of about 10 to 25 nucleotides in length.

Embodiment 24 is the method of any one of embodiments 1-23, wherein the capture domain comprises a unique nucleotide sequence.

Embodiment 25 is the method of any one of embodiments 1-24, wherein the analyte is a protein.

Embodiment 26 is the method of embodiment 25, wherein the protein is an intracellular protein.

Embodiment 27 is the method of embodiment 25, wherein the protein is an extracellular protein.

Embodiment 28 is the method of any one of embodiments 25-27, wherein the analyte binding moiety is an antibody or an antigen-binding fragment thereof.

Embodiment 29 is the method of any one of embodiments 1-28, wherein the analyte capture agent further comprises a linker, wherein the linker is disposed between the analyte binding moiety and the analyte binding moiety barcode.

Embodiment 30 the method of embodiment 29, wherein the linker is a cleavable linker.

Embodiment 31 is the method of embodiment 30, wherein the cleavable linker is a photo-cleavable linker or an enzyme-cleavable linker.

Embodiment 32 is he method of any one of embodiments 1-31, wherein the method further comprises determining a sequence of (i) all or a part of the sequence of the analyte binding moiety barcode or a complement thereof, and (ii) all or a part of the sequence of the spatial barcode or a complement thereof, and using the determined sequence of (i) and (ii) to identify a location of the analyte in the biological sample.

Embodiment 33 is the method of embodiment 32, wherein the determining comprises sequencing (i) all or a part of the sequence of the analyte binding moiety barcode or a complement thereof, and (ii) all or a part of the sequence of the spatial barcode or a complement thereof.

Embodiment 34 is the method of embodiment 33, wherein the sequencing comprises high throughput sequencing.

Embodiment 35 is the method of any one of embodiments 1-34, wherein the biological sample is a tissue sample.

Embodiment 36 is the method of embodiment 35, wherein the tissue sample is a fixed tissue sample.

Embodiment 37 is the method of embodiment 36, wherein the fixed tissue sample comprises a formalin-fixed paraffin-embedded (FFPE) tissue sample.

Embodiment 38 is the method of embodiment 35, wherein the tissue sample is a fresh tissue sample or a frozen tissue sample.

Embodiment 39 is a kit comprising: (a) an array, wherein the array comprises a plurality of capture probes, wherein a capture probe of the plurality of capture probes comprises a spatial barcode and a capture domain; and (b) a plurality of analyte capture agents, wherein an analyte capture agent comprises an analyte binding moiety that binds specifically to an analyte in a biological sample, an analyte binding moiety barcode, and an analyte capture sequence, wherein the capture domain, the analyte capture sequence, or both are reversibly blocked with one or more blocking probes.

Embodiment 40 is the kit of embodiment 39, wherein the capture domain is reversibly blocked with a blocking probe of the one or more blocking probes.

Embodiment 41 is the kit of embodiment 39, wherein the analyte capture sequence is reversibly blocked with a blocking probe of the one or more blocking probes.

Embodiment 42 is the kit of embodiment 39, wherein the capture domain is reversibly blocked with a first blocking probe of the one or more blocking probes and the analyte capture sequence is reversibly blocked with a second blocking probe of the one or more blocking probes.

Embodiment 43 is the kit of any one of embodiments 39-42, wherein the kit further comprises an enzyme.

Embodiment 44 is the kit of embodiment 43, wherein the enzyme is an endonuclease.

Embodiment 45 is the kit of embodiment 44, wherein a blocking probe of the one or more blocking probes comprises one or more inosine nucleotides and the endonuclease is endonuclease V.

Embodiment 46 is the kit of embodiment 44, wherein a blocking probe of the one or more blocking probes comprises one or more abasic sites and the endonuclease is endonuclease IV.

Embodiment 47 is the kit of embodiment 43, wherein a blocking probe of the one or more blocking probes comprises a uracil and the enzyme is a uracil-specific excision reagent (USER).

Embodiment 48 is the kit of embodiment 47, wherein the blocking probe comprises a poly(U) sequence, one or more RNA bases, one or more LNA bases, and combinations thereof.

Embodiment 49 is the kit of any one of embodiments 39-42, wherein a blocking probe of the one or more blocking probes, when hybridized to the analyte capture sequence or the capture domain, comprises one or more mismatched nucleotides.

Embodiment 50 is the kit of embodiment 49, wherein the one or more mismatched nucleotides in the blocking probe hybridized to the analyte capture sequence or the capture domain are positioned, after the fourth nucleotide from a 5′ end of the blocking probe and before the last four nucleotides at the 3′ end of the blocking probe.

Embodiment 51 is the kit of embodiment 50, wherein one or more mismatched nucleotides in the blocking probe hybridized to the analyte capture sequence or the capture domain are positioned, after the sixth nucleotide from the 5′ end of the blocking probe and before the last six nucleotides at the 3′ end of the blocking probe.

Embodiment 52 is the kit of any one of embodiments 45-51, wherein the blocking probe has a length of about 8 to about 24 nucleotides.

Embodiment 53 is the kit of any one of embodiments 39-52, wherein the capture domain comprises a nucleotide sequence of about 10 to 25 nucleotides in length.

Embodiment 54 is the kit of any one of embodiments 39-53, wherein the capture domain comprises a unique nucleotide sequence.

Embodiment 55 is the kit of any one of embodiments 39-54, wherein the analyte is a protein.

Embodiment 56 is the kit of embodiment 55, wherein the protein is an intracellular protein.

Embodiment 57 is the kit of embodiment 55, wherein the protein is an extracellular protein.

Embodiment 58 is the kit of any one of embodiments 55-57, wherein the analyte binding moiety is an antibody or an antigen-binding fragment thereof.

Embodiment 59 is the kit of any one of embodiments 39-58, wherein the analyte capture agent further comprises a linker, wherein the linker is disposed between the analyte binding moiety and the analyte binding moiety barcode.

Embodiment 60 is the kit of embodiment 59, wherein the linker is a cleavable linker.

Embodiment 61 is the kit of embodiment 60, wherein the cleavable linker is a photo-cleavable linker or an enzyme-cleavable linker.

What is claimed is:

1. A method for reducing non-specific binding of an analyte capture sequence to a capture domain on a spatial array comprising:
(a) providing an array, wherein the array comprises a plurality of capture probes, wherein a capture probe of the plurality of capture probes comprises: (i) a spatial barcode and (ii) a capture domain sequence that hybridizes to the analyte capture sequence;
(b) providing a plurality of analyte capture agents to a biological sample, wherein an analyte capture agent of the plurality of analyte capture agents comprises: an analyte binding moiety that specifically binds to an analyte in the biological sample, and an oligonucleotide comprising: (i) an analyte binding moiety barcode and (ii) an analyte capture sequence that hybridizes to the capture domain, and
reversibly blocking the capture domain, the analyte capture sequence, or both, with one or more blocking probes; and
(c) releasing the one or more blocking probes from the analyte capture sequence, the capture domain, or both, and allowing the analyte capture sequence to hybridize to the capture domain, thereby reducing non-specific binding of the analyte capture sequence to the capture domain on the spatial array.

2. The method of claim 1, wherein the blocking probe specifically blocks the analyte capture sequence of the analyte capture agent and not the capture domain of the capture probe on the array.

3. The method of claim 1, further comprising fixing the biological sample, and optionally wherein the fixing comprises methanol, and staining the biological sample, and optionally, wherein the staining comprises immunofluorescence.

4. The method of claim 1, wherein, prior to the providing in step (b), contacting the plurality of analyte capture agents with the one or more blocking probes.

5. The method of claim 1, wherein the capture domain is reversibly blocked with a blocking probe of the one or more blocking probes.

6. The method of claim 1, wherein the analyte capture sequence is reversibly blocked with a blocking probe of the one or more blocking probes.

7. The method of claim 1, wherein the capture domain is reversibly blocked with a first blocking probe of the one or more blocking probes and the analyte capture sequence is reversibly blocked with a second blocking probe of the one or more blocking probes.

8. The method of claim 1, wherein releasing in step (c) of the one or more blocking probes comprises use of an enzyme, optionally, wherein the enzyme is an endonuclease, wherein:
(i) a blocking probe of the one or more blocking probes comprises one or more inosine nucleotides and the endonuclease is endonuclease V; or
(ii) a blocking probe of the one or more blocking probes comprises one or more abasic sites and the endonuclease is endonuclease IV.

9. The method of claim 8, wherein a blocking probe of the one or more blocking probes comprises a uracil and the enzyme is a uracil-specific excision reagent.

10. The method of claim 9, wherein the blocking probe comprises a poly(U) sequence, one or more RNA bases, one or more LNA bases, and combinations thereof.

11. The method of claim 1, wherein the biological sample is disposed on the array.

12. The method of claim 1, wherein the biological sample is disposed on a substrate.

13. The method of claim 12, further comprising aligning the substrate comprising the biological sample with the array, such that at least a portion of the biological sample is aligned with at least a portion of the array.

14. The method of claim 1, wherein a blocking probe of the one or more blocking probes, when hybridized to the analyte capture sequence and/or the capture domain, comprises one or more mismatched nucleotides, and the releasing comprises increasing the temperature of the biological sample.

15. The method of claim 14, wherein the one or more mismatched nucleotides of the blocking probe hybridized to the analyte capture sequence and/or the capture domain are positioned after the fourth nucleotide from a 5′ end of the blocking probe and before the last four nucleotides at the 3′ end of the blocking probe.

16. The method of claim 14, wherein the one or more mismatched nucleotides in the blocking probe hybridized to the analyte capture sequence and/or the capture domain are positioned after the sixth nucleotide from the 5′ end of the blocking probe and before the last six nucleotides at the 3′ end of the blocking probe.

17. The method of claim 1, wherein a blocking probe has a length of about 8 nucleotides to about 24 nucleotides.

18. The method of claim 1, wherein the releasing of the one or more blocking probes comprises washing the biological sample.

19. The method of claim 1, wherein the method further comprises permeabilizing the biological sample.

20. The method of claim 1, wherein the capture domain comprises a nucleotide sequence of about 10 nucleotides to about 25 nucleotides in length.

21. The method of claim 1, wherein the capture domain comprises a unique nucleotide sequence.

22. The method of claim 1, wherein the analyte comprises a protein.

23. The method of claim 1, wherein the analyte binding moiety comprises an antibody or an antigen-binding fragment thereof.

24. The method of claim 1, wherein the analyte binding moiety barcode identifies the analyte specifically bound by the analyte binding moiety.

25. The method of claim 1, wherein the analyte capture agent further comprises a linker, wherein the linker is disposed between the analyte binding moiety and the analyte binding moiety barcode.

26. The method of claim 25, wherein the linker is a cleavable linker, and optionally, wherein the cleavable linker is a photo-cleavable linker or an enzyme cleavable linker.

27. The method of claim 1, further comprising determining a sequence of (i) the sequence of the analyte binding moiety barcode, or a complement thereof, and (ii) the sequence of the spatial barcode, or a complement thereof, and using the determined sequences of (i) and (ii) to determine a location of the analyte in the biological sample.

28. The method of claim 1, wherein prior to step (c) the biological sample is stained with hematoxylin and eosin or immunofluorescence, and imaged.

29. The method of claim 1, wherein the biological sample is a tissue sample, a fixed tissue sample, a formalin-fixed paraffin-embedded tissue sample, or a fresh-frozen tissue sample.

30. The method of claim 1, wherein the biological sample is a tissue section.


SEQUENCE LISTING (26 sequences)

SEQ ID NO: 1 - DNA, 12 bp, synthetic construct
  source 1..12: mol_type = other DNA
ttgctaggaccg

SEQ ID NO: 2 - DNA, 14 bp, synthetic construct
  source 1..14: mol_type = other DNA
ttgctaggaccggc

SEQ ID NO: 3 - DNA, 16 bp, synthetic construct
  source 1..16: mol_type = other DNA
ttgctaggaccggcct

SEQ ID NO: 4 - DNA, 18 bp, synthetic construct
  source 1..18: mol_type = other DNA
ttgctaggaccggcctta

SEQ ID NO: 5 - DNA, 22 bp, synthetic construct
  source 1..22: mol_type = other DNA
ttgctaggaccggccttaaagc

SEQ ID NO: 6 - DNA, 22 bp, synthetic construct
  source 1..22: mol_type = other DNA
gctttaaggccggtcctagcaa

SEQ ID NO: 7-10 - skipped (4 entries)

SEQ ID NO: 11 - DNA, 12 bp, synthetic construct
  source 1..12: mol_type = other DNA
  modified_base 5: mod_base = OTHER; note = uracil
ttgctaggaccg

SEQ ID NO: 12 - DNA, 16 bp, synthetic construct
  source 1..16: mol_type = other DNA
  modified_base order(7,12): mod_base = i
ttgctangaccngcct

SEQ ID NO: 13 - DNA, 22 bp, synthetic construct
  source 1..22: mol_type = other DNA
  modified_base order(7,12,13,20): mod_base = i
ttgctangaccnnccttaangc

SEQ ID NO: 14-15 - skipped (2 entries)

SEQ ID NO: 16 - DNA, 14 bp, synthetic construct
  source 1..14: mol_type = other DNA
  modified_base order(5,11): mod_base = OTHER; note = uracil
ttgctaggactggc

SEQ ID NO: 17 - DNA, 22 bp, synthetic construct
  source 1..22: mol_type = other DNA
  modified_base order(5,12,16): mod_base = OTHER; note = uracil
ttgctaggacctgccttaaagc

SEQ ID NO: 18 - skipped

SEQ ID NO: 19 - DNA, 14 bp, synthetic construct
  source 1..14: mol_type = other DNA
  modified_base order(6,9): mod_base = OTHER; note = uracil
gccggtcctagcaa

SEQ ID NO: 20 - DNA, 22 bp, synthetic construct
  source 1..22: mol_type = other DNA
  modified_base order(5,10,14,17): mod_base = OTHER; note = uracil
gctttaaggtcggtcctagcaa

SEQ ID NO: 21 - skipped

SEQ ID NO: 22 - DNA, 23 bp, synthetic construct
  source 1..23: mol_type = other DNA
  misc_feature 1..22: note = RNA
  misc_feature 23: note = DNA
  modified_base 23: mod_base = OTHER; note = inverted thymine
ttgctaggaccggccttaaagct

SEQ ID NO: 23 - DNA, 23 bp, synthetic construct
  source 1..23: mol_type = other DNA
  misc_feature order(1,3..11,13..20,22): note = RNA
  misc_feature order(2,12,21,23): note = DNA
  modified_base 23: mod_base = OTHER; note = inverted thymine
  modified_base order(2,12,21): mod_base = OTHER; note = locked nucleic acid
ttgctaggaccggccttaaagct

SEQ ID NO: 24 - DNA, 23 bp, synthetic construct
  source 1..23: mol_type = other DNA
  misc_feature order(1,3..6,8..11,13..15,17..20,22): note = RNA
  misc_feature order(2,7,12,16,21,23): note = DNA
  modified_base 23: mod_base = OTHER; note = inverted thymine
  modified_base order(2,7,12,16,21): mod_base = OTHER; note = locked nucleic acid
ttgctaggaccggccttaaagct

SEQ ID NO: 25 - DNA, 23 bp, synthetic construct
  source 1..23: mol_type = other DNA
  misc_feature order(1,3,4,6,7,9,10,12,13,15,16,18,19,21,22): note = RNA
  misc_feature order(2,5,8,11,14,17,20,23): note = DNA
  modified_base 23: mod_base = OTHER; note = inverted thymine
  modified_base order(2,5,8,11,14,17,20): mod_base = OTHER; note = locked nucleic acid
ttgctaggaccggccttaaagct

SEQ ID NO: 26 - DNA, 23 bp, synthetic construct
  source 1..23: mol_type = other DNA
  misc_feature order(1,3..11,13..20,22): note = RNA
  misc_feature order(2,12,21,23): note = DNA
  modified_base 23: mod_base = OTHER; note = inverted thymine
  modified_base order(2,12,21): mod_base = OTHER; note = locked nucleic acid
ttgctaggaccggccttaaagct